(19) United States
(12) Patent Application Publication
(10) Pub. No.: US 2009/0170919 A1
(43) Pub. Date: Jul. 2, 2009

(54) METHODS FOR TREATMENT OF INFLAMMATORY DISEASES

(75) Inventor: Elliott FARBER, North Mankato, MN (US)

Correspondence Address: HOGAN & HARTSON LLP;IP GROUP, COLUMBIA SQUARE, 555 THIRTEENTH STREET, N.W., WASHINGTON, DC, 20004, US

(73) Assignee: ALWYN COMPANY, INC., Lake Crystal, MN (US)

(21) Appl. No.: 12/396,424

(22) Filed: Mar. 2, 2009

Related U.S. Application Data
(63) Continuation of application No. 11/266,251, filed on Nov. 4, 2005.
Application No. 11/266,251 is a continuation-in-part of application No. 09/991,283, filed on Nov. 13, 2001, now abandoned.
Application No. 09/991,283 is a continuation-in-part of application No. 09/758,696, filed on Jan. 11, 2001, now Pat. No. 6,673,826.
Application No. 09/758,696 is a continuation-in-part of application No. 09/570,120, filed on May 12, 2000, now Pat. No. 6,531,500.
Application No. 09/570,120 is a continuation-in-part of application No. 09/360,095, filed on Jul. 23, 1999, now Pat. No. 6,281,236.

(51) Int. Cl. A61K 31/415

(52) U.S. Cl. 514/390

(57) ABSTRACT

An improved method of treating skin diseases comprises applying to the skin of a patient suffering such a skin disease an allantoin-containing composition in a therapeutically effective quantity. The allantoin-containing composition is an oil-in-water emulsion that includes allantoin and an emulsifier system that includes at least one emulsifier that is either an anionic emulsifier or a nonionic emulsifier. The pH of the composition used in a method according to the present invention is from approximately 3.0 to approximately 6.0; a narrower pH range can be used, varying with each embodiment of the composition.

### CROSS-REFERENCES

This application is a continuation-in-part of application Ser. No. 09/991,283, entitled “Methods for Treatment of Inflammatory Diseases,” filed Nov. 13, 2001 by Elliott Farber, which in turn is a continuation-in-part of application Ser. No. 09/758,696, entitled “Methods for Treatment of Inflammatory Diseases,” filed Jan. 11, 2001 by Elliott Farber, which in turn is a continuation-in-part application of application Ser. No. 09/570,120, entitled “Methods for Treatment of Inflammatory Diseases,” filed May 12, 2000 by Elliott Farber, which in turn is a continuation-in-part application of application Ser. No. 09/360,095, entitled “Oil-in-Water Emulsion With Improved Stability,” filed Jul. 23, 1999 by Elliott Farber, now U.S. Pat. No. 6,281,236, issued Aug. 28, 2001. All three of these prior applications are hereby incorporated in their entirety by this reference.

### BACKGROUND OF THE INVENTION

General Background and State of the Art: The present invention is directed to improved methods of treating inflammatory skin disease.

Inflammatory skin disease, particularly chronic inflammatory skin disease, is still a major source of morbidity. Such inflammatory skin diseases are disfiguring and cause severe physical and psychological harm to patients, disrupting their quality of life substantially. Such diseases include decubitus ulcers, pressure ulcers, diabetic ulcers, epidermolysis bullosa, and milia, as well as other conditions affecting the skin and having an inflammatory component such as eczema, urticaria, atopic dermatitis, contact dermatitis, arthritis, gout, and lupus erythematosus. Such skin diseases tend to be chronic and difficult to treat, particularly in patients with poor circulation or other underlying disease states.

Among the most difficult of these diseases to treat is epidermolysis bullosa. Epidermolysis bullosa occurs in newborns and infants and causes severe inflammation, blistering, and scarring.

Accordingly, there is a need for an improved method of treating these inflammatory skin diseases. Such a method should be effective in a wide variety of skin diseases, and should be suitable for use together with other treatment modalities. It should be well tolerated by the patients without side effects. This is particularly important because many of these diseases have an underlying allergic component that makes their treatment difficult and may prevent the use of a number of previously known agents.

### INVENTION SUMMARY

An improved method of treating such skin diseases comprises applying to the skin of a patient suffering such a skin disease an allantoin-containing composition in a therapeutically effective quantity.

The allantoin-containing composition comprises an oil-in-water emulsion including at least one emulsifier and can contain other ingredients, such as a chelating agent to bind metal ions that might accelerate degradation of the composition. A particularly preferred chelating agent is EDTA. The EDTA can be added in various acid or salt forms depending on the pH of the composition, such as EDTA itself, disodium EDTA, or tetrasodium EDTA.

In one embodiment of the invention, the allantoin-containing composition comprises an oil-in-water emulsion comprising:

(1) allantoin;

(2) an emulsifier system including:
(a) an acidic wax; and
(b) an anionic emulsifier that is substantially hydrophilic and is soluble in water; and

(3) an acid to adjust the pH of the composition to a value within the range of from about 3.0 to about 6.0. Preferably, the pH of the composition is from about 4.5 to about 5.8.

Acidic waxes are those waxes having acidic groups that can be neutralized with alkaline materials such as hydroxides, alkoxides, unprotonated amines, and/or salts of strong bases and weak acids, such as sodium acetate. Upon neutralization, such waxes can act as emulsifiers or coemulsifiers. Preferred acidic waxes include beeswax, carnauba wax, candelilla wax, siliconyl beeswax, siliconyl carnauba wax, and synthetic acidic waxes. A particularly preferred acidic wax is beeswax.

The emulsifier can be selected from the group consisting of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, and sodium lauryl sulfate. Preferably, the emulsifier is sodium lauryl sulfate.

In another embodiment, the allantoin-containing composition comprises an oil-in water emulsion comprising:

(1) allantoin;

(2) an emollient component comprising:
(a) lanolin oil;
(b) cetyl alcohol;
(c) stearyl alcohol; and
(d) cod liver oil;

(3) butylated hydroxytoluene;

(4) an emulsifier system comprising at least one nonionic emulsifier that is an ethoxylated ether or an ethoxylated ester whose carbon chain length ranges from 8 to 22 carbon atoms; and

(5) at least one acid selected from the group consisting of:
(a) an organic acid of from 2 to 22 carbon atoms; and
(b) an inorganic acid selected from the group consisting of hydrochloric acid, sulfuric acid, and phosphoric acid to adjust the pH from about 3.0 to about 6.0. Preferably, the pH of the composition is from about 4.5 to about 5.8.

In yet another embodiment, the allantoin-containing composition comprises an oil in-water emulsion comprising:

(1) allantoin;

(2) an emulsifier system including at least one nonionic emulsifier that is an ethoxylated ether or an ethoxylated ester whose carbon chain length ranges from 8 to 22 carbon atoms; and

(3) an acid to adjust the pH of the composition to a value within the range of from about 3.0 to about 6.0. Preferably, the pH of the composition is from about 4.5 to about 5.8.

In still another embodiment, the allantoin-containing composition comprises an oil-in-water emulsion comprising:

(1) allantoin; and

(2) an emulsifier system comprising:
(a) an acidic anionic polymer; and
(b) a polyethylene glycol ester of stearic acid.

The pH of the composition is adjusted to a value within a range of from about 3.0 to about 6.0. Preferably, the pH of the composition is from about 5.0 to about 6.0.

The composition can further comprise a carbohydrate polymer selected from the group consisting of galactoarabinan, polygalactose, and polyarabinose; preferably, the carbohydrate polymer is galactoarabinan.

In yet another alternative embodiment, the allantoin-containing composition comprises an oil-in-water emulsion comprising:

(1) allantoin; and

(2) an emulsifier system comprising:
(a) an acidic anionic polymer; and
(b) an anionic emulsifier that is substantially hydrophilic and is soluble in water.

The pH of the composition is adjusted to a value in a range from about 3.0 to about 6.0. Preferably, the pH of the composition is from about 5.0 to about 6.0.

The anionic emulsifier can be selected from the group consisting of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, and sodium lauryl sulfate. Preferably, the anionic emulsifier is sodium lauryl sulfate.

Typically, the acidic anionic polymer is carboxypolymethylene.

Preferably, in this embodiment, the composition further comprises a carbohydrate polymer selected from the group consisting of galactoarabinan, polygalactose, and polyarabinose. More preferably, the carbohydrate polymer is galactoarabinan.

In yet another alternative, the allantoin-containing composition comprises an oil-in-water emulsion comprising:

(1) allantoin; and

(2) an emulsifier system comprising:
(a) an acidic anionic polymer; and
(b) a nonionic emulsifier that is an ethoxylated ether or an ethoxylated ester whose carbon chain length ranges from 8 to 22 carbon atoms, wherein the pH of the composition is from about 3.0 to about 6.0. Preferably, the pH of the composition is from about 5.0 to about 6.0.

The acidic anionic polymer is preferably carboxypolymethylene as described above.

Preferably, in this embodiment as well, the composition further comprises a carbohydrate polymer selected from the group consisting of galactoarabinan, polygalactose and polyarabinose. More preferably, the carbohydrate polymer is galactoarabinan.

In this embodiment, the emulsifier system can further comprise glyceryl stearate.

In yet another embodiment of a method according to the present invention, the ethoxylated ether or ethoxylated ester is omitted in the composition. In this embodiment, the composition comprises an oil-in-water emulsion comprising:

(1) allantoin;

(2) an emulsifier system comprising an acidic anionic polymer;

(3) an organic or inorganic base to adjust the pH to a value in a range of from about 3.0 to about 6.0. Preferably, the pH of the composition is from about 5.0 to about 5.5.

Preferably, the base is triethanolamine, and the acidic anionic polymer is a carboxypolymethylene polymer as described above.

Yet another embodiment of a method according to the present invention uses an allantoin-containing composition comprising an oil-in-water emulsion comprising:

(1) allantoin;

(2) an emulsifier system comprising:
(a) cetyl alcohol; and
(b) stearic acid; and

(3) a weak organic base to adjust the pH to a value within a range of from about 3.0 to about 6.0. Preferably, the pH of the composition is from about 5.0 to about 5.8.

Typically, the weak organic base is triethanolamine.

Still another embodiment of a method according to the present invention uses an allantoin-containing composition comprising an oil-in-water emulsion comprising:

(1) allantoin; and

(2) an emulsifier system comprising:
(a) sodium stearoyl lactylate;
(b) sodium isostearoyl lactylate;
(c) optionally, triethanolamine stearate; and
(d) optionally, at least one nonionic emulsifier selected from the group consisting of a nonionic emulsifier that is an ethoxylated ether or an ethoxylated ester whose carbon chain length ranges from 8 to 22 carbon atoms; and

(3) an acid to adjust the pH to a value within a range of from about 3.0 to about 6.0. Preferably, the pH of the composition is from about 5.0 to about 5.8.

Typically, the acid is citric acid.

Still another embodiment of a method according to the present invention uses an allantoin-containing composition comprising an oil-in-water emulsion comprising:

(1) allantoin; and

(2) an emulsifier system comprising at least one polyethyleneglycol ether of cetearyl alcohol; and

(3) an acid to adjust the pH of the composition to a value within a range of from about 3.0 to about 6.0. Preferably, the pH of the composition is from about 5.0 to about 5.8.

Typically, the acid is citric acid.

In polyethylene glycol ethers of cetearyl alcohol suitable for use in compositions in this embodiment of a method according to the present invention, the number of ethylene glycol moieties can range from 6 to 40, e.g., R(OCH₂CH₂)₂₅OH where R is CH₃(CH₂)₁₆₋₁₈. In one preferred composition suitable for use in this embodiment of a method according to the present invention, the emulsifier system comprises both ceteareth-25 and ceteareth-6, i.e., polyethylene glycol ethers of cetearyl alcohol with 25 and 6 ethylene glycol units respectively.

Yet another embodiment of a method according to the present invention uses an allantoin-containing composition comprising an oil-in-water emulsion comprising:

(1) allantoin; and

(2) an emulsifier system comprising:
(a) a polyethylene glycol ester of stearic acid; and
(b) glyceryl stearate; and

(3) an acid to adjust the pH of the composition to a value within a range of from about 3.0 to about 6.0. Preferably, the pH of the composition is from about 5.0 to about 5.8.

Typically, the number of ethylene glycol moieties in the polyethylene glycol ester of stearic acid is from 25 to 100. Two preferred polyethylene glycol esters of stearic acid for use in compositions for an embodiment of a method according to the present invention are PEG-40 stearate and PEG-100 stearate, with 40 and 100 ethylene glycol moieties respectively. A particularly preferred polyethylene glycol ester of stearic acid is PEG-100 stearate.

Typically, the acid is citric acid.

in yet another alternative embodiment, the allantoin-containing composition comprises an oil-in-water emulsion comprising:

(1) allantoin;

(2) a carbohydrate polymer; and

(3) an emulsifier system comprising:
(a) an acidic wax; and
(b) an anionic emulsifier that is substantially hydrophilic and is soluble in water.

The pH of the composition is between about 3.0 and about 6.0. Preferably, the pH of the composition is from about 5.0 to about 6.0.

The carbohydrate polymer in this embodiment is as described above.

Typically, the anionic emulsifier that is substantially hydrophilic and soluble in water is selected from the group consisting of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, and sodium lauryl sulfate. Preferably, the anionic emulsifier is sodium lauryl sulfate.

The acidic wax in this embodiment is as described above. Typically, the acidic wax is beeswax, carnauba wax, candelilla wax, siliconyl beeswax, siliconyl carnauba wax, or a synthetic acidic wax. Preferably, the acidic wax is beeswax.

In yet another alternative embodiment, the allantoin-containing composition comprises an oil-in-water emulsion comprising:

(1) allantoin in a concentration of at least about 2.5% and;

(2) an emulsifier system comprising:
(a) an acidic wax; and
(b) an anionic emulsifier that is substantially hydrophilic and is soluble in water.

The pH of the composition is in a range from about 3.0 to about 6.0.

Typically, the anionic emulsifier that is substantially hydrophilic and soluble in water is selected from the group consisting of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, and sodium lauryl sulfate. Preferably, the anionic emulsifier is sodium lauryl sulfate.

The acidic wax in this embodiment is as described above. Typically, the acidic wax is beeswax, carnauba wax, candelilla wax, siliconyl beeswax, siliconyl carnauba wax, or a synthetic acidic wax. Preferably, the acidic wax is beeswax.

The composition can further comprise citric acid to adjust the pH.

In all of these alternative embodiments, the composition can further comprise additional ingredients if they are not already included.

For example, the composition can further comprise an emollient component. The emollient component can comprise at least one emollient selected from the group consisting of lanolin oil, cetyl alcohol, stearyl alcohol, and cod liver oil.

The composition can further comprise an antioxidant such as butylated hydroxytoluene or butylated hydroxyanisole.

The composition can further comprise a preservative component. The preservative component can comprise at least one preservative selected from the group consisting of methylparaben, propylparaben, and diazolidinyl urea. Other preservatives can alternatively be used

The composition can further comprise a chelating agent. A preferred chelating agent is tetrasodium EDTA.

The composition can further comprise a solvent component. The solvent component can comprise at least one solvent selected from the group consisting of ethylene glycol, propylene glycol, butylene glycol, and glycerin. Preferably, the solvent component is propylene glycol.

The skin condition or disease to be treated can be one of epidermolysis bullosa, decubitus ulcers, pressure ulcers, diabetic ulcers, and milia or another inflammatory disease or condition, such as conditions affecting the skin and having an inflammatory component such as eczema, urticaria, atopic dermatitis, contact dermatitis, arthritis, gout, and lupus erythematosus. An important skin condition or disease that is treated by methods according to the present invention is epidermolysis bullosa.

Methods according to the present invention can further comprise administering an additional therapeutic agent in a therapeutically effective quantity. The additional therapeutic agent can be selected from the group consisting of steroids, nonsteroidal anti-inflammatory agents, leukotriene antagonists, and monoclonal antibodies.

### BRIEF DESCRIPTION OF THE DRAWINGS

These and other features, aspects, and advantages of the present invention will become better understood with reference to the following description, appended claims, and accompanying drawings where:

FIGS. 1(a) and 1(b) are pictures of the right foot of a first patient (A.B.) before the use of the cream of Example 2 from two different views, showing the severity of the disease;

FIG. 2 is a picture of the right foot of the patient A.B. after two months of use of the cream of Example 2, showing considerable improvement;

FIGS. 3(a) and 3(b) are pictures of the right foot of the patient A.B. after 12 months of use of the cream of Example 2, showing substantial improvement and clearing of the lesions;

FIGS. 4(a), 4(b), and 4(c) are additional pictures of the right foot of the patient A.B. after 12 months of use of the cream of Example 2, again showing substantial improvement and clearing of the lesions;

FIGS. 5(a) and 5(b) are pictures of the buttocks area of the patient A.B. before the use of the cream (FIG. 5(a)) and after 2 weeks of use of the cream of Example 2 (FIG. 5(b)), showing substantial improvement and clearing of the lesions;

FIGS. 6(a) and 6(b) are pictures of the facial area of the patient A.B. before the use of the cream (FIG. 6(a)) and after 3 months of use of the cream of Example 2 (FIG. 6(b)), showing substantial improvement, fading, and clearing of the lesions;

FIG. 7 is a photograph of a second patient (C.D.) before commencement of the use of the allantoin-containing skin cream of Example 2;

FIG. 8 is a photograph of patient C.D. after 8 weeks of use of the allantoin-containing skin cream of Example 2, showing substantial improvement of the lesions;

FIG. 9(a) is a photograph of the back area of patient C.D. before commencement of the use of the allantoin-containing skin cream of Example 2;

FIG. 9(b) is another photograph of the back area of patient C.D. before commencement of the use of the allantoin-containing skin cream of Example 2;

FIG. 10(a) is a photograph of the upper back area of patient C.D. after 2 weeks of use of the allantoin-containing skin cream of Example 2, showing considerable improvement;

FIG. 10(b) is a photograph of the upper back area of patient C.D. after 8 weeks of use of the allantoin-containing skin cream of Example 2, showing continued improvement evidenced by fading of the lesions;

FIG. 11(a) is a photograph of the upper leg area of patient C.D. before commencement of the use of the allantoin-containing skin cream of Example 2;

FIG. 11(b) is a photograph of the lower leg area of patient C.D. before commencement of the use of the allantoin-containing skin cream of Example 2;

FIG. 11(c) is a photograph of the legs of patient C.D. after 2 weeks of use of the allantoin-containing skin cream of Example 2, showing substantial improvement; and

FIG. 11(d) is a photograph of the legs of patient C.D. after 8 weeks of use of the allantoin-containing skin cream of Example 2, showing continuing improvement.

### DETAILED DESCRIPTION OF THE PREFERRED EMBODIMENTS

I have unexpectedly found that a stabilized oil-in-water emulsion containing allantoin plus other ingredients provides a high degree of relief for inflammatory skin conditions characterized by ulceration, inflammation, or blistering of the skin.

In general, a method of treating such a skin condition or disease comprises applying to the skin an allantoin-containing composition in a therapeutically effective amount. The allantoin-containing composition comprises an oil-in-water emulsion as described below.

The conditions that can be treated include, but are not limited to, decubitus ulcers, pressure ulcers, diabetic ulcers, epidermolysis bullosa, and milia, as well as other conditions affecting the skin and having an inflammatory component such as eczema, urticaria, atopic dermatitis, contact dermatitis, arthritis, gout, and lupus erythematosus. Additional conditions that can be treated include acne, alopecia, carcinomas, psoriasis, rosacea, miliaria, skin infections, post-operative care of incisions, post-operative skin care following any variety of plastic surgery procedures, skin care following radiation treatment, care of dry, cracked or aged skin and skin lines. As described below in Examples 19-20, methods of the present invention are particularly suited for the treatment of epidermolysis bullosa.

The allantoin-containing composition comprises an oil-in-water emulsion including at least one emulsifier and can contain other ingredients, such as a chelating agent to bind metal ions that might accelerate degradation of the composition. A particularly preferred chelating agent is EDTA. The EDTA can be added in various acid or salt forms depending on the pH of the composition, such as EDTA itself, disodium EDTA, or tetrasodium EDTA.

In one embodiment of the present invention, the allantoin-containing composition comprises an oil-in-water emulsion comprising:

(1) allantoin;

(2) an emulsifier system including:
(i) an acidic wax; and
(ii) an anionic emulsifier that is substantially hydrophilic and is soluble in water; and

(3) an acid to adjust the pH of the emulsion to a value in the range of from about 3.0 to about 6.0.

Preferably, the pH of the emulsion is from about 4.5 to about 5.8.

Acidic waxes are those waxes having acidic groups that can be neutralized with alkaline materials such as hydroxides, alkoxides, unprotonated amines, and/or salts of strong bases and weak acids, such as sodium acetate. Upon neutralization, such waxes can act as emulsifiers or coemulsifiers. Preferred acidic waxes include beeswax, carnauba wax, candelilla wax, siliconyl beeswax, siliconyl carnauba wax, and synthetic acidic waxes. Examples of synthetic acidic waxes are syncrowaxes marketed by Croda, Inc. A particularly preferred acidic wax is beeswax.

The anionic emulsifier is typically one of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, or sodium lauryl sulfate. A particularly preferred anionic emulsifier is sodium lauryl sulfate.

The acid used to adjust the pH can be an organic acid, an inorganic acid, or a mixture of both.

Preferred organic acids include organic acids whose carbon chain length ranges from 2 to 22 carbon atoms and can be monocarboxylic, dicarboxylic, or tricarboxylic acids. The acids can be aliphatic or aromatic. Particularly preferred organic acids include citric acid, ascorbic acid, glycolic acid, benzoic acid, and salicylic acid. A most particularly preferred organic acid is citric acid.

Typically, the inorganic acid is a strong acid. It can be a monoprotic, diprotic, or triprotic acid. Particularly preferred inorganic acids include hydrochloric acid, sulfuric acid, or phosphoric acid.

For the embodiments described above, the composition can further include other ingredients. For example, the composition can include an emollient component for smoothness. The emollient component can include at least one of lanolin oil, cetyl alcohol, stearyl alcohol, and cod liver oil. Preferably, the emollient component comprises all of lanolin oil, cetyl alcohol, stearyl alcohol, and cod liver oil.

The composition can also include an antioxidant to prevent rancidity of ingredients such as cod liver oil. A preferred antioxidant is butylated hydroxytoluene (BHT). Other antioxidants such as butylated hydroxyanisole (BHA) can be used, alternatively or in addition to BHT.

The composition can further include a solvent component. Typically, the solvent component is one or more of ethylene glycol, propylene glycol, butylene glycol, and glycerin. Preferably, the solvent component is propylene glycol.

The composition can further include a chelating agent to bind metal ions that might accelerate degradation of the composition. A particularly preferred chelating agent is tetrasodium EDTA.

The composition can further include herbal extracts. The herbal extracts can include one or more of St. John's wort extract, witch hazel extract, chamomile extract, and arnica extract. The composition can include all of St. John's wort extract, witch hazel extract, chamomile extract, and arnica extract. However, typically, in compositions used in methods according to the present invention, herbal extracts are omitted.

The composition can further include a preservative such as at least one of methylparaben, ethylparaben, propylparaben, butylparaben, or phenoxyethanol. Preferably, the composition comprises methlylparaben and propylparaben as preservatives.

The composition can further include fragrance. The use of fragrance is well known in the art of over-the-counter drug formulation, and many suitable fragrances are known in the art. The stability and function of the composition is not altered by the presence or absence of fragrance. In many alternatives, it may be desirable to avoid the use of fragrance which may trigger allergic reactions in patients predisposed to such reactions.

The composition can further include other ingredients, such as proteins, humectants, other preservatives, essential oils, other vitamins, colorants, hydroxyacids, other plant extracts, sunscreens, sodium hyaluronate, lipids, fatty acids, thickeners, panthenol, and the like. The use of such components is conventional in the over-the-counter drug art. Typical sunscreens are octyl methoxycinnamate and benzophenone-3.

The following discussion describes ranges, preferred concentrations, and optimum concentrations for preferred compositions when the pH of the composition is from about 4.5 to about 5.8 useful in this embodiment of methods according to the present invention. For this and other ranges, preferred concentrations, and optimum concentrations of specific ingredients for other embodiments as given below, all percentages are weight percentages unless otherwise specified.

Water can comprise from about 50.0% to about 90.0% of the composition. Preferably, water comprises from about 55.0% to about 75.0% of the composition. In one alternative, in which the optimum concentration of allantoin is about 1.50% of the composition, the optimum concentration of water is about 68.68% of the composition. In another alternative, in which the optimum concentration of allantoin is about 9.00% of the composition, the optimum concentration of water is about 61.18% of the composition.

Sodium lauryl sulfate, as a 30% solution, can comprise from about 0.5% to about 2.5% of the composition. Preferably, sodium lauryl sulfate comprises from about 1.0% to about 2.5% of the composition. An optimum concentration of sodium lauryl sulfate in the composition is about 1.90%.

Propylene glycol can comprise from about 2.0% to about 9.0% of the composition. Preferably, propylene glycol comprises from about 3.0% to about 6.0% of the composition. An optimum concentration of propylene glycol is about 5.30% of the composition.

Tetrasodium EDTA can comprise from about 0.05% to about 0.50% of the composition. Preferably, tetrasodium EDTA comprises from about 0.1% to about 0.30% of the composition. An optimum concentration of tetrasodium EDTA is about 0.15% of the composition.

Citric acid can comprise from about 0.05% to about 0.50% of the composition. A preferred concentration of citric acid is from about 0.08% to about 0.35% of the composition. An optimum concentration of citric acid is about 0.12% of the composition.

Lanolin oil can comprise from about 5.0% to about 15.0% of the composition. Preferably, lanolin oil comprises from about 8.0% to about 12.0% of the composition. An optimum concentration of lanolin oil is about 10.60% of the composition.

Cetyl alcohol can comprise from about 3.0% to about 10.0% of the composition. A preferred concentration of cetyl alcohol is from about 3.5% to about 7.5% of the composition. An optimum concentration of cetyl alcohol is about 4.20% of the composition.

Stearyl alcohol can comprise from about 1.0% to about 5.0% of the composition. A preferred concentration of stearyl alcohol is from about 1.0% to about 3.0% of the composition. An optimum concentration of stearyl alcohol is about 2.00% of the composition.

The acidic wax, such as beeswax can comprise from about 0.5% to about 2.5% of the composition. A preferred concentration of the acidic wax, such as beeswax, is from about 1.0% to about 2.5% of the composition. An optimum concentration of the acidic wax, such as beeswax, is about 1.90% of the composition.

Cod liver oil can comprise from about 1.0% to about 7.0% of the composition. Preferably, cod liver oil comprises from about 1.0% to about 4.0% of the composition. An optimum concentration of cod liver oil is about 2.00% of the composition.

Butylated hydroxytoluene can comprise from about 0.1% to about 1.0% of the composition. Preferably, butylated hydroxytoluene comprises from about 0.2% to about 0.8% of the composition. An optimum concentration of butylated hydroxytoluene is about 0.50% of the composition.

St. John's wort extract can comprise from about 0.05% to about 0.5% of the composition. Preferably, St. John's wort extract comprises from about 0.05% to about 0.15% of the composition. An optimum concentration of St. John's wort extract is about 0.10% of the composition.

Witch hazel extract can comprise from about 0.05% to about 0.5% of the composition. Preferably, witch hazel extract comprises from about 0.05% to about 0.15% of the composition. An optimum concentration of witch hazel extract is about 0.10% of the composition.

Chamomile extract can comprise from about 0.05% to about 0.50% of the composition. A preferred concentration of chamomile extract is from about 0.05% to about 0.15% of the composition. An optimum concentration of chamomile extract is about 0.10% of the composition.

Arnica extract can comprise from about 0.05% to about 0.50% of the composition. Preferably, arnica extract comprises from about 0.05% to about 0.15% of the composition. An optimum concentration of arnica extract is about 0.10% of the composition.

Methylparaben can comprise from about 0.10% to about 0.50% of the composition. A preferred concentration of methylparaben is from about 0.15% to about 0.40% of the composition. An optimum concentration of methylparaben is about 0.30% of the composition.

Propylparaben can comprise from about 0.10% to about 0.50% of the composition. A preferred concentration of propylparaben is from about 0.10% to about 0.30% of the composition. An optimum concentration of propylparaben is about 0.25% of the composition.

Allantoin can comprise from about 0.50% to about 10.0% of the composition. In one alternative, a preferred concentration of allantoin is from about 1.0% to about 2.0% of the composition. In this alternative, the optimum concentration of allantoin is about 150% of the composition. In another alternative, an optimum concentration of allantoin is about 9.00% of the composition.

If present, fragrance can comprise from about 0.05% to about 0.50% of the composition. Preferably, fragrance comprises from about 0.10% to about 0.30% of the composition. If present, an optimum concentration of fragrance is about 0.20% of the composition. As indicated above, in many embodiments it is desirable to omit fragrance to avoid the possibility of allergic reactions.

In another embodiment, the composition comprises an oil-in-water emulsion comprising:

(1) allantoin; and

(2) an emulsifier system including at least one nonionic emulsifier that is an ethoxylated ether or an ethoxylated ester whose carbon chain length ranges from 8 to 22 carbon atoms, wherein the pH of the composition is from about 3.0 to about 6.0. Preferably, the pH of the composition is from about 4.5 to about 5.8.

The composition used in this embodiment of the method can further include other ingredients as described above. For example, the composition can further include:

(1) an emollient component comprising at least one emollient selected from the group consisting of lanolin oil, cetyl alcohol, stearyl alcohol, and cod liver oil;

(2) an antioxidant such as butylated hydroxytoluene or butylated hydroxyanisole as described above;

(3) at least one herbal extract selected from the group consisting of St. John's wort extract, witch hazel extract, chamomile extract, and arnica extract;

(4) a preservative component comprising at least one preservative selected from the group consisting of methylparaben, propylparaben, and diazolidinyl urea;

(5) tetrasodium EDTA; and

(6) a solvent component comprising at least one solvent selected from the group consisting of ethylene glycol, propylene glycol, butylene glycol, and glycerin.

In yet another embodiment of a method according to the present invention, the composition comprises an oil-in-water emulsion comprising:

(1) allantoin;

(2) an emollient component comprising:
(a) lanolin oil;
(b) cetyl alcohol;
(c) stearyl alcohol; and
(d) cod liver oil;

(3) butylated hydroxytoluene;

(4) an emulsifier system comprising at least one nonionic emulsifier that is an ethoxylated ether or an ethoxylated ester whose carbon chain length ranges from 8 to 22 carbon atoms; and

(5) at least one acid selected from the group consisting of:
(a) an organic acid of from 2 to 22 carbon atoms; and
(b) an inorganic acid selected from the group consisting of hydrochloric acid, sulfuric acid, and phosphoric acid to adjust the pH from about 3.0 to about 6.0. Preferably, the pH of the composition is from about 4.5 to about 5.8.

The composition can further include other ingredients as described above. For example, the composition can further include:

(1) at least one herbal extract selected from the group consisting of St. John's wort extract, witch hazel extract, chamomile extract, and arnica extract;

(2) a preservative component comprising at least one preservative selected from the group consisting of methylparaben, propylparaben, and diazolidinyl urea;

(3) tetrasodium EDTA; and

(4) a solvent component comprising at least one solvent selected from the group consisting of ethylene glycol, propylene glycol, butylene glycol, and glycerin.

In still another embodiment of the method, the allantoin-containing composition comprises an oil-in-water emulsion comprising:

(1) allantoin; and

(2) an emulsifier system comprising:
(a) an acidic anionic polymer; and
(b) a polyethylene glycol ester of stearic acid.

The pH of the composition is adjusted to a value in the range of from about 3.0 to about 6.0. Preferably, the pH of the composition is from about 5.0 to about 6.0. The pH is adjusted with sodium hydroxide or other base as required.

The acidic anionic polymer is preferably a carboxypolymethylene polymer. Such Li polymers are marketed under the brand names “Carbomer” and “Carbopol.” A suitable carboxypolymethylene polymer is marketed by B.F. Goodrich under the brand name “Carbomer.” This is a slightly cross-linked polyacrylic acid that is from 1% to 2% cross-linked by allylsucrose or allylpentaerythritol with the polyacrylic acid. The resulting molecular weight range of this polymer is from about 2×10⁶ daltons to about 1×10⁹ daltons. The average molecular weight of this polymer is about 4×10⁶ daltons.

Preferably, the concentration of the carboxypolymethylene polymer is from about 0.5% to about 2% of the composition.

The composition can further comprise a carbohydrate polymer. Typically, the carbohydrate polymer is selected from the group consisting of galactoarabinan, polygalactose, and polyarabinose. Preferably, the carbohydrate polymer is galactoarabinan. Galactoarabinan is derived from trees of the genus Larix (larch) and is a hemicellulosic product easily extractable by water in a pure form. The molecular weight of the galactoarabinan is about 20,000. Galactoarabinan has been consumed by humans in common foods such as carrots, tomatoes, maple syrup, soybeans, and wheat flour, among others. A suitable source of galactoarabinan is Larex, Inc. (White Bear Lake, Minn.). Typically, the composition contains from about 1% to about 25% of galactoarabinan. Preferably, the composition contains from about 2% to about 10% of the carbohydrate polymer.

The composition used in this embodiment of a method according to the present invention can further include other ingredients. For example, the composition can include an emollient component for smoothness. The emollient component can include at least one emollient selected from the group consisting of lanolin oil, cetyl alcohol, stearyl alcohol, and cod liver oil.

The composition can also include an antioxidant to prevent rancidity of ingredients such as cod liver oil. A preferred antioxidant is butylated hydroxytoluene (BHT). Other antioxidants such as butylated hydroxyanisole (BHA) can be used, alternatively or in addition to BHT.

The composition can further include a solvent component. Typically, the solvent component can include at least one solvent selected from the group consisting of ethylene glycol, propylene glycol, glycerin, and butylene glycol. Preferably, the solvent component is propylene glycol.

The composition can further include a preservative component. The preservative component can include at least one preservative selected from the group consisting of methylparaben, propylparaben, and diazolidinyl urea. Preferably, the preservative component comprises methylparaben, propylparaben, and diazolidinyl urea.

The composition can further include fragrance. The use of fragrance is well known in the cosmetic art and in the art of over-the-counter drug formulation, and many suitable fragrances are known in the art. The stability and function of the cream is not altered by the presence or absence of fragrance.

Optionally, the composition can further include herbal extracts. The herbal extracts can include one or more of St. John's wort extract, witch hazel extract, chamomile extract, and arnica extract. However, these herbal extracts are typically omitted in the composition used in this embodiment of a method according to the present invention.

The composition can optionally further include other components, such as proteins, humectants, other preservatives, essential oils, other vitamins, colorants, hydroxyacids, other plant extracts, chelators, sunscreens, sodium hyaluronate, lipids, fatty acids, thickeners, panthenol, and the like. The use of such components is conventional in the cosmetic art and in the over-the-counter drug art. Typical sunscreens are octyl methoxycinnamate and benzophenone-3.

The following discussion describes ranges, preferred concentrations and optimum concentrations for preferred compositions useful in this embodiment of the present invention when the pH of the composition is from about 5.0 to about 6.0.

Water can comprise from about 50.0% to about 90.0% of the composition. Preferably, water comprises from about 60.0% to about 85.0% of the composition. In one alternative, in which the optimum concentration of allantoin is about 1.50% of the composition, the optimum concentration of water is about 69.95% of the composition. In another alternative, in which the optimum concentration of water is about 9.00% of the composition, the optimum concentration of water is about 62.45% of the composition.

The carboxypolymethylene polymer can comprise from about 0.30% to about 3.0% of the composition. Preferably, the carboxypolymethylene polymer comprises from about 0.50% to about 2.0% of the composition. An optimum concentration of the carboxypolymethylene polymer is about 0.85% of the composition.

Propylene glycol can comprise from about 2.0% to about 9.0% of the composition. Preferably, propylene glycol comprises from about 4.0% to about 7.0% of the composition. An optimum concentration of propylene glycol is about 5.70% of the composition.

PEG-100 stearate can comprise from about 0.25% to about 2.5% of the composition. Preferably, PEG-100 stearate comprises from about 0.50% to about 2.0% of the composition. An optimum concentration of PEG-100 stearate is about 1.50% of the composition.

Lanolin oil can comprise from about 5.0% to about 15.0% of the composition. Preferably, lanolin oil comprises from about 8.0% to about 12.0% of the composition. An optimum concentration of lanolin oil is about 10.60% of the composition.

Cetyl alcohol can comprise from about 1.0% to about 8.0% of the composition. A preferred concentration of cetyl alcohol is from about 2.0% to about 7.0% of the composition. An optimum concentration of cetyl alcohol is about 4.20% of the composition.

Stearyl alcohol can comprise from about 0.5% to about 6.0% of the composition. A preferred concentration of stearyl alcohol is from about 0.75% to about 5.0% of the composition. An optimum concentration of stearyl alcohol is about 1.50% of the composition.

Cod liver oil can comprise from about 1.0% to about 7.0% of the composition. Preferably, cod liver oil comprises from about 1.0% to about 4.0% of the composition. An optimum concentration of cod liver oil is about 2.00% of the composition.

Butylated hydroxytoluene can comprise from about 0.10% to about 1.0% of the composition. Preferably, butylated hydroxytoluene comprises from about 0.20% to about 0.80% of the composition. An optimum concentration of butylated hydroxytoluene is about 0.50% of the composition.

Methylparaben can comprise from about 0.10% to about 0.50% of the composition. A preferred concentration of methylparaben is from about 0.15% to about 0.40% of the composition. An optimum concentration of methylparaben is about 0.30% of the composition.

Propylparaben can comprise from about 0.10% to about 0.50% of the composition. Preferably, propylparaben comprises from about 0.15% to about 0.40% of the composition. An optimum concentration of propylparaben is about 0.25% of the composition.

Diazolidinyl urea can comprise from about 0.05% to about 0.25% of the composition. Preferably, diazolidinyl urea comprises from about 0.10% to about 0.20% of the composition. An optimum concentration of diazolidinyl urea is about 0.15% of the composition.

Allantoin can comprise from about 0.50% to about 10.0% of the composition. In one alternative, a preferred concentration of allantoin is from about 1.0% to about 2.0% of the composition. In this alternative, the optimum concentration of allantoin is about 1.50% of the composition. In another alternative, an optimum concentration of allantoin is about 9.00% of the composition.

Fragrance can comprise from about 0.05% to about 0.50% of the composition. Preferably, fragrance comprises from about 0.10% to about 0.40% of the composition. An optimum concentration of fragrance is about 0.20% of the composition. As indicated above, fragrance can be omitted, and it may be desirable to omit fragrance in circumstances in which the composition is intended for use on sensitive individuals or individuals who may undergo an allergic reaction to fragrance.

Triethanolamine can comprise from about 0.05% to about 3.0% of the composition to adjust the pH. A preferred concentration of triethanolamine is from about 0.20% to about 2.0% of the composition. An optimum concentration of triethanolamine is about 0.80% of the composition.

In another alternative embodiment of a method according to the present invention, the emulsifier of the composition can be an anionic emulsifier that is substantially hydrophilic and is soluble in water. In this embodiment, the anionic emulsifier replaces the polyethylene glycol ester of stearic acid. The composition further includes the acidic anionic polymer such as carboxypolymethylene. Optionally, but preferably, the composition includes the carbohydrate polymer such as galactoarabinan.

The anionic emulsifier that is substantially hydrophilic and soluble in water can be selected from the group consisting of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, and sodium lauryl sulfate. A particularly preferred anionic emulsifier is sodium lauryl sulfate.

Commercially available preparations of sodium lauryl sulfate contain sufficient excess sodium hydroxide so that they have a pH of about 10.0. This sodium hydroxide can be used to adjust the pH when the anionic emulsifier is sodium lauryl sulfate; in this alternative, no additional alkali may be needed. When another anionic emulsifier is used, additional alkali may be required to adjust the pH.

In yet another alternative embodiment of a method according to the present invention, the emulsifier system of the composition used in the method comprises the acidic anionic polymer as described above and a nonionic emulsifier that is an ethoxylated ether or an ethoxylated ester whose carbon chain length ranges from 8 to 22 carbon atoms.

Preferably, the acidic anionic polymer is carboxypolymethylene as described above.

This alternative of the composition used in the method can further include glyceryl stearate in the emulsifier system.

The composition has a pH from about 3.0 to 6.0, adjusted as necessary, typically with an acid. The acid can be an organic acid, an inorganic acid, or a mixture of both. Preferably, the composition has a pH from about 5.0 to about 6.0.

This embodiment of the composition can further comprise a carbohydrate polymer such as galactoarabinan as described above.

In the composition, preferred organic acids include organic acids whose carbon chain length ranges from 2 to 22 carbon atoms and can be monocarboxylic, dicarboxylic, or tricarboxylic acids. The acids can be aliphatic or aromatic. Particularly preferred organic acids include citric acid, ascorbic acid, glycolic acid, lactic acid, benzoic acid, and salicylic acid. A most particularly preferred organic acid is citric acid.

Typically, in the composition, the inorganic acid is a strong acid. It can be a monoprotic, diprotic, or triprotic acid. Particularly preferred inorganic acids include hydrochloric acid, sulfuric acid, and phosphoric acid.

The composition can further include other ingredients as described above, including an emollient component, an antioxidant, a solvent component, a chelating agent, herbal extracts, a preservative, and fragrance.

In particular, the composition can further include at least one of:

(1) an emollient component comprising at least one emollient selected from the group consisting of lanolin oil, cetyl alcohol, stearyl alcohol, and cod liver oil;

(2) an antioxidant such as butylated hydroxytoluene or butylated hydroxyanisole;

(3) at least one herbal extract selected from the group consisting of St. John's wort extract, witch hazel extract, chamomile extract, and arnica extract;

(4) a preservative component comprising at least one preservative selected from the group consisting of methylparaben, propylparaben and diazolidinyl urea;

(5) tetrasodium EDTA; and

(6) a solvent component comprising at least one solvent selected from the group consisting of ethylene glycol, propylene glycol, butylene glycol, and glycerin.

The composition can further include other components, such as proteins, humectants, other preservatives, essential oils, other vitamins, colorants, hydroxyacids, other plant extracts, sunscreens, sodium hyaluronate, lipids, fatty acids, thickeners, panthenol, and the like. The use of such components is conventional in the cosmetic art and in the over-the-counter drug art. Typical sunscreens are octyl methoxycinnamate and benzophenone-3.

In yet another embodiment of a method according to the present invention, the emulsifier system of the composition used in the method comprises the acidic anionic polymer described above; one example of this acidic anionic polymer is marketed as Carbomer. In this embodiment, the pH is adjusted with an organic or inorganic base to a value within a range of from about 3.0 to about 6.0. Preferably, the pH of the composition is from about 5.0 to about 5.5. A preferred organic base is triethanolamine. A preferred inorganic base is sodium hydroxide. In general, it is preferred to use an organic base such as triethanolamine.

The composition used in this embodiment of the method can further comprise other ingredients. For example, the composition can further comprise a solvent component. Typically, the solvent component includes at least one solvent selected from the group consisting of ethylene glycol, propylene glycol, glycerin, or butylene glycol. Preferably, the solvent component is propylene glycol.

The composition can further comprise an emollient component. The emollient component can comprise at least one solvent selected from the group consisting of lanolin oil, cetyl alcohol, and cod liver oil. Preferably, the emollient component comprises all of lanolin oil, cetyl alcohol, and cod liver oil.

The composition can also include an antioxidant to prevent rancidity of ingredients such as cod liver oil. A preferred antioxidant is butylated hydroxytoluene (BHT). Other antioxidants such as butylated hydroxyanisole (BHA) can be used, alternatively or in addition to BHT.

The composition can further comprise a preservative component. The preservative component can comprise at least one preservative selected from the group consisting of methylparaben and propylparaben. Preferably, the preservative component comprises both methylparaben and propylparaben.

The composition can further include fragrance as described above. The stability and function of the cream is not altered by the presence or absence of fragrance. As indicated above, it may be desirable to omit fragrance in some cases.

The following discussion describes, ranges, preferred concentrations and optimum concentrations for preferred compositions with a pH of from about 5.0 to about 5.5 according to this embodiment of the method of the present invention.

Water can comprise from about 50.0% to about 90.0% of the composition. Preferably, water comprises from about 60.0% to about 80.0% of the composition. In one alternative, in which the optimum concentration of allantoin is about 1.50% of the composition, the optimum concentration of water is about 73.55% of the composition. In another alternative, in which the optimum concentration of water is about 9.00% of the composition, the optimum concentration of water is about 66.05% of the composition.

The carboxypolymethylene polymer can comprise from about 0.40% to about 3.0% of the composition. Preferably, the carboxypolymethylene polymer comprises from about 0.5% to about 2.0% of the composition. An optimum concentration of the carboxypolymethylene polymer is about 1.00% of the composition.

Propylene glycol can comprise from about 2.0% to about 9.0% of the composition. Preferably, the propylene glycol comprises from about 4.0% to about 7.0% of the composition. An optimum concentration of the propylene glycol is about 5.70% of the composition.

Lanolin oil can comprise from about 5.0% to about 15.0% of this embodiment of the composition. Preferably, lanolin oil comprises from about 8.0% to about 12.0% of this embodiment of the composition. An optimum concentration of lanolin oil is about 10.00% of this embodiment of the composition.

Cetyl alcohol can comprise from about 1.0% to about 8.0% of the composition. Preferably, cetyl alcohol comprises from about 2.0% to about 7.0% of the composition. An optimum concentration of cetyl alcohol is about 3.00% of the composition.

Cod liver oil can comprise from about 1.0% to about 7.0% of the composition. Preferably, cod liver oil comprises from about 1.0% to about 4.0% of the composition. An optimum concentration of cod liver oil is about 2.00% of the composition.

Butylated hydroxytoluene can comprise from about 0.10% to about 1.0% of the composition. Preferably, butylated hydroxytoluene comprises from about 0.30% to about 0.80% of the composition. An optimum concentration of butylated hydroxytoluene is about 0.50% of the composition.

Methylparaben can comprise from about 0.10% to about 0.50% of the composition. Preferably, methylparaben comprises from about 0.15% to about 0.40% of the composition. An optimum concentration of methylparaben is about 0.30% of the composition.

Propylparaben can comprise from about 0.10% to about 0.50% of the composition. Preferably, propylparaben comprises from about 0.15% to about 0.40% of the composition. An optimum concentration of propylparaben is about 0.25% of the composition.

Allantoin can comprise from about 0.50% to about 10.0% of the composition. In one alternative, a preferred concentration of allantoin is from about 1.0% to about 2.0% of the composition. In this alternative, the optimum concentration of allantoin is about 1.50% of the composition. In another alternative, an optimum concentration of allantoin is about 9.00% of the composition.

Fragrance, if present, can comprise from about 0.05% to about 0.50% of the composition. Preferably, if present, fragrance comprises from about 0.10% to about 0.40% of the composition. An optimum concentration of fragrance, if present, is about 0.20% of the composition.

Triethanolamine, as a 95% solution, can comprise from about 0.05% to about 3.0% of the composition to adjust the pH to a value in the range of from about 5.0 to about 5.5. Preferably, triethanolamine comprises from about 0.20% to about 2.0% of the composition to adjust the pH as indicated. An optimum concentration of triethanolamine is about 0.80% of the composition to adjust the pH as indicated.

Yet another embodiment of a method according to the present invention employs a composition comprising an oil-in-water emulsion comprising:

(1) allantoin; and

(2) an emulsifier system comprising:
(a) cetyl alcohol; and
(b) stearic acid.

In this embodiment, the pH of the composition is adjusted to a value within a range of from about 3.0 to about 6.0 by addition of a quantity of a weak organic base. Preferably, the pH of the composition is from about 5.0 to about 5.8. The weak organic base can be an amine-containing base such as ethanolamine, diethanolamine, or triethanolamine. A preferred organic base is triethanolamine.

The composition used in this embodiment of a method according to the present invention can further comprise other ingredients. For example, the composition can further comprise a solvent component. Typically, the solvent component includes at least one solvent selected from the group consisting of ethylene glycol, propylene glycol, glycerin, and butylene glycol. Preferably, the solvent component is propylene glycol.

The composition can further comprise an emollient component. The emollient component can comprise at least one solvent selected from the group consisting of lanolin oil, cetyl alcohol, and cod liver oil. Preferably, the emollient component comprises all of lanolin oil, cetyl alcohol, and cod liver oil.

The composition can also include an antioxidant. A preferred antioxidant is butylated hydroxytoluene. Other antioxidants such as butylated hydroxyanisole (BHA) can be used, alternatively or in addition to BHT.

The composition can further comprise a preservative component. The preservative component can comprise at least one preservative selected from the group consisting of methylparaben and propylparaben. Preferably, the preservative component comprises both methylparaben and propylparaben.

The composition can further include fragrance as described above. The stability and function of the cream is not altered by the presence or absence of fragrance. As indicated above, it may be desirable to omit fragrance in some cases.

The following discussion describes ranges, preferred concentrations and optimum concentrations for preferred compositions with a pH of from about 5.0 to about 5.8 according to this embodiment of a method of the present invention.

Water can comprise from about 50.0% to about 90.0% of the composition. Preferably, water comprises from about 60.0% to about 85.0% of the composition. In one alternative, in which the optimum concentration of allantoin is about 1.50% of the composition, the optimum concentration of water is about 71.70% of the composition. In another alternative, in which the optimum concentration of allantoin is about 9.00% of the composition, the optimum concentration of water is about 64.20% of the composition.

Propylene glycol can comprise from about 2.0% to about 9.0% of the composition. Preferably, propylene glycol comprises from about 4.0% to about 7.0% of the composition. An optimum concentration of propylene glycol is about 5.70% of the composition.

Triethanolamine can comprise from about 0.2% to about 4.0% of the composition. Preferably, triethanolamine comprises from about 0.5% to about 3.0% of the composition. An optimum concentration of triethanolamine is about 1.25% of the composition.

Lanolin oil can comprise from about 5.0% to about 15.0% of the composition. Preferably, lanolin oil comprises from about 8.0% to about 12.0% of the composition. An optimum concentration of lanolin oil is about 10.60% of the composition.

Cetyl alcohol can comprise from about 1.0% to about 7.0% of the composition. Preferably, cetyl alcohol comprises from about 2.0% to about 6.0% of the composition. An optimum concentration of cetyl alcohol is about 3.50% of the composition.

Stearic acid can comprise from about 0.50% to about 5.0% of the composition. Preferably, stearic acid comprises from about 1.0% to about 4.0% of the composition. An optimum concentration of stearic acid is about 2.50% of the composition.

Cod liver oil can comprise from about 1.0% to about 7.0% of the composition. Preferably, cod liver oil comprises from about 1.50% to about 5.0% of the composition. An optimum concentration of cod liver oil is about 2.00% of the composition.

Butylated hydroxytoluene can comprise from about 0.1% to about 1.0% of the composition. Preferably, butylated hydroxytoluene comprises from about 0.2% to about 0.8% of the composition. An optimum concentration of butylated hydroxytoluene is about 0.5% of the composition.

Methylparaben can comprise from about 0.10% to about 0.50% of the composition. Preferably, methylparaben comprises from about 0.15% to about 0.40% of the composition. An optimum concentration of methylparaben is about 0.30% of the composition.

Propylparaben can comprise from about 0.10% to about 0.50% of the composition. Preferably, propylparaben comprises from about 0.15% to about 0.40% of the composition. An optimum concentration of propylparaben is about 0.25% of the composition.

Allantoin can comprise from about 0.50% to about 10.0% of the composition. In one alternative, a preferred concentration of allantoin is from about 1.0% to about 2.0% of the composition. In this alternative, the optimum concentration of allantoin is about 1.50% of the composition. In another alternative, an optimum concentration of allantoin is about 9.00% of the composition.

If present, fragrance can comprise from about 0.05% to about 0.50% of the composition. Preferably, fragrance comprises from about 0.10% to about 0.40% of the composition. An optimum concentration of fragrance is about 0.20% of the composition.

Still another embodiment of a method according to the present invention employs a composition comprising an oil-in-water emulsion comprising:

(1) allantoin; and

(2) an emulsifier system comprising:
(a) sodium stearoyl lactylate;
(b) sodium isostearoyl lactylate;
(c) optionally, triethanolamine stearate;
(d) optionally, at least one nonionic emulsifier selected from the group consisting of a nonionic emulsifier that is an ethoxylated ether or an ethoxylated ester whose carbon chain length ranges from 8 to 22 carbon atoms.

Sodium stearoyl I1612actylate is the sodium salt of the stearic acid ester of lactyl lactate. Sodium isostearoyl lactylate is the sodium salt of the isostearic acid ester of lactyl lactate.

In the composition used in this embodiment of a method according to the present invention, the composition further comprises an acid to adjust the pH to a value in a range of from about 3.0 to about 6.0. Preferably, the composition has a pH of from about 5.0 to about 5.8. The acid can be an inorganic or an organic acid as described above. Preferably, the acid is a weak organic acid. Most preferably, the acid is citric acid.

The composition can further comprise other ingredients. For example, the composition can further comprise a solvent component. Typically, the solvent component includes at least one solvent selected from the group consisting of ethylene glycol, propylene glycol, glycerin, and butylene glycol. Preferably, the solvent component is propylene glycol.

The composition can further comprise an emollient component. The emollient component can comprise at least one emollient selected from the group consisting of lanolin oil, cetyl alcohol, and cod liver oil. Preferably, the emollient component comprises all of lanolin oil, cetyl alcohol, and cod liver oil.

The composition can also include an antioxidant. A preferred antioxidant is butylated hydroxytoluene. Other antioxidants such as butylated hydroxyanisole (BHA) can be used, alternatively or in addition to BHT.

The composition can further comprise a chelator component. Preferably, the chelator component is tetrasodium ethylenediaminetetraacetic acid.

The composition can further comprise a preservative component. The preservative component can comprise at least one preservative selected from the group consisting of methylparaben and propylparaben. Preferably, the preservative component comprises both methylparaben and propylparaben.

The composition can further include fragrance as described above. The stability and function of the cream is not altered by the presence or absence of fragrance. As indicated above, it may be desirable to omit fragrance in some cases.

The following discussion describes ranges, preferred concentrations and optimum concentrations for preferred compositions with a pH of from about 5.0 to about 5.8 according to this embodiment of a method according to the present invention.

Water can comprise from about 50.0% to about 90.0% of the composition. Preferably, water comprises from about 60.0% to about 80.0% of the composition. In one alternative, in which the optimum concentration of allantoin is about 1.50% of the composition, the optimum concentration of water is about 73.72% of the composition. In another alternative, in which the optimum concentration of allantoin is about 9.00% of the composition, the optimum concentration of water is about 66.22% of the composition.

Propylene glycol can comprise from about 2.0% to about 9.0% of the composition. Preferably, propylene glycol comprises from about 4.0% to about 7.0% of the composition. An optimum concentration of propylene glycol is about 5.70% of the composition.

Citric acid can comprise from about 0.05% to about 0.50% of the composition. Preferably, citric acid comprises from about 0.10% to about 0.40% of the composition. An optimum concentration of citric acid is about 0.18% of the composition.

Sodium stearoyl lactylate can comprise from about 0.30% to about 3.0% of the composition. Preferably, sodium stearoyl lactylate comprises from about 0.50% to about 2.50% of the composition. An optimum concentration of sodium stearoyl lactylate is about 1.00% of the composition.

Sodium isostearoyl lactylate can comprise from about 0.05% to about 1.0% of the composition. Preferably, sodium isostearoyl lactylate comprises from about 0.10% to about 0.70% of the composition. An optimum concentration of sodium isostearoyl lactylate is about 0.25% of the composition.

Tetrasodium EDTA can comprise from about 0.05% to about 0.25% of the composition. Preferably, tetrasodium EDTA comprises from about 0.10% to about 0.20% of the composition. An optimum concentration of tetrasodium EDTA is about 0.15% of the composition.

Lanolin oil can comprise from about 5.0% to about 15.0% of the composition. Preferably, lanolin oil comprises from about 8.0% to about 12.0% of the composition. An optimum concentration of lanolin oil is about 10.60% of the composition.

Cetyl alcohol can comprise from about 1.0% to about 8.0% of the composition. Preferably, cetyl alcohol comprises from about 2.0% to about 7.0% of the composition. An optimum concentration of cetyl alcohol is about 3.80% of the composition.

Cod liver oil can comprise from about 1.0% to about 7.0% of the composition. Preferably, cod liver oil comprises from about 1.0% to about 4.0% of the composition. An optimum concentration of cod liver oil is about 2.00% of the composition.

Butylated hydroxytoluene can comprise from about 0.10% to about 1.0% of the composition. Preferably, butylated hydroxytoluene comprises from about 0.20% to about 0.80% of the composition. An optimum concentration of butylated hydroxytoluene is about 0.50% of the composition.

Methylparaben can comprise from about 0.10% to about 0.50% of the composition. Preferably, methylparaben comprises from about 0.15% to about 0.40% of the composition. An optimum concentration of methylparaben is about 0.30% of the composition.

Propylparaben can comprise from about 0.10% to about 0.50% of the composition. Preferably, propylparaben comprises from about 0.15% to about 0.40% of the composition. An optimum concentration of propylparaben is about 0.25% of the composition.

Allantoin can comprise from about 0.50% to about 10.0% of the composition. In one alternative, a preferred concentration of allantoin is from about 1.0% to about 2.0% of the composition. In this alternative, the optimum concentration of allantoin is about 1.50% of the composition. In another alternative, an optimum concentration of allantoin is about 9.00% of the composition.

If present, fragrance can comprise from about 0.05% to about 0.50% of the composition. Preferably, fragrance comprises from about 0.10% to about 0.40% of the composition. An optimum concentration of fragrance is about 0.20% of the composition.

Still another embodiment of a method according to the present invention uses a composition comprising an oil-in-water emulsion comprising:

(1) allantoin; and

(2) an emulsifier system comprising at least one polyethyleneglycol ether of cetearyl alcohol.

In polyethylene glycol ethers of cetearyl alcohol suitable for use in compositions according to this embodiment of methods of the present invention, the number of ethylene glycol moieties can range from 6 to 40, e.g., R(OCH₂CH₂)₂₅OH where R is CH₃(CH₂)_{16.18}. In one preferred embodiment of compounds of the present invention, the emulsifier system comprises both ceteareth-25 and ceteareth-6, i.e., polyethylene glycol ethers of cetearyl alcohol with 25 and 6 ethylene glycol units respectively.

In this embodiment of a method according to the present invention, the composition further comprises an acid to adjust the pH to a value within a range of from about 3.0 to about 6.0. Preferably, the pH of the composition is from about 5.0.to about 5.8. The acid can be an inorganic or an organic acid as described above. Preferably, the acid is a weak organic acid. Most preferably, the acid is citric acid.

The composition can further comprise other ingredients. For example, the composition can further comprise a solvent component. Typically, the solvent component is selected from the group consisting of ethylene glycol, propylene glycol, glycerin, or butylene glycol. Preferably, the solvent component is propylene glycol.

The composition can further comprise a chelator component. Preferably, the chelator component is tetrasodium ethylenediaminetetraacetic acid.

The composition can further comprise an emollient component. The emollient component can comprise at least one emollient selected from the group consisting of lanolin oil, cetyl alcohol, stearyl alcohol, and cod liver oil. Preferably, the emollient component comprises all of lanolin oil, cetyl alcohol, stearyl alcohol, and cod liver oil.

The composition can also further comprise an antioxidant. A preferred antioxidant is butylated hydroxytoluene. Other antioxidants such as butylated hydroxyanisole (BHA) can be used, alternatively or in addition to BHT.

The composition can further comprise a preservative component. The preservative component can comprise at least one preservative selected from the group consisting of methylparaben, propylparaben, and diazolidinyl urea. Preferably, the preservative component comprises all of methylparaben, propylparaben, and diazolidinyl urea.

The composition can further include fragrance as described above. The stability and function of the cream is not altered by the presence or absence of fragrance. As indicated above, it may be desirable to omit fragrance in some cases.

The following discussion describes ranges, preferred concentrations and optimum concentrations for preferred compositions with a pH of from about 5.0 to about 5.8 according to this embodiment of a method according to the present invention.

Water can comprise from about 50.0% to about 90.0% of the composition. Preferably, water comprises from about 55.0% to about 75.0% of the composition. In one alternative, in which the optimum concentration of allantoin is about 1.50% of the composition, the optimum concentration of water is about 66.33% of the composition. In another alternative, in which the optimum concentration of allantoin is about 9.00% of the composition, the optimum concentration of water is about 58.83% of the composition.

Propylene glycol can comprise from about 2.0% to about 9.0% of the composition. Preferably, propylene glycol comprises from about 4.2% to about 7.0% of the composition. An optimum concentration of propylene glycol is about 5.70% of the composition.

Tetrasodium EDTA can comprise from about 0.05% to about 0.50% of the composition. Preferably, tetrasodium EDTA comprises from about 0.10% to about 0.30% of the composition. An optimum concentration of tetrasodium EDTA is about 0.15% of the composition.

Ceteareth-25 can comprise from about 0.50% to about 4.0% of the composition. Preferably, ceteareth-25 comprises from about 2.0% to about 3.5% of the composition. An optimum concentration of ceteareth-25 is about 2.60% of the composition.

Citric acid can comprise from about 0.04% to about 0.40% of the composition. Preferably, citric acid comprises from about 0.10% to about 0.30% of the composition. An optimum concentration of citric acid is about 0.12% of the composition.

Lanolin oil can comprise from about 5.0% to about 15.0% of the composition. Preferably, lanolin oil comprises from about 8.0% to about 12.0% of the composition. An optimum concentration of lanolin oil is about 10.60% of the composition.

Cetyl alcohol can comprise from about 3.0% to about 10.0% of the composition. Preferably, cetyl alcohol comprises from about 3.5% to about 7.5% of the composition. An optimum concentration of cetyl alcohol is about 4.30% of the composition.

Stearyl alcohol can comprise from about 1.0% to about 5.0% of the composition. Preferably, stearyl alcohol comprises from about 2.0% to about 4.0% of the composition. An optimum concentration of stearyl alcohol is about 3.50% of the composition.

Ceteareth-6 can comprise from about 0.5% to about 4.0% of the composition. Preferably, ceteareth-6 comprises from about 1.0% to about 3.0% of the composition. An optimum concentration of ceteareth-6 is about 1.80% of the composition.

Cod liver oil can comprise from about 1.0% to about 7.0% of the composition. Preferably, cod liver oil comprises from about 1.0% to about 4.0% of the composition. An optimum concentration of cod liver oil is about 2.00% of the composition.

Butylated hydroxytoluene can comprise from about 0.10% to about 1.0% of the composition. Preferably, butylated hydroxytoluene comprises from about 0.20% to about 0.80% of the composition. An optimum concentration of butylated hydroxytoluene is about 0.50% of the composition.

Methylparaben can comprise from about 0.10% to about 0.50% of the composition. Preferably, methylparaben comprises from about 0.15% to 0.40% of the composition. An optimum concentration of methylparaben is about 0.30% of the composition.

Propylparaben can comprise from about 0.10% to about 0.50% of the composition. Preferably, propylparaben comprises from about 0.15% to about 0.40% of the composition. An optimum concentration of propylparaben is about 0.25% of the composition.

Diazolidinyl urea can comprise from about 0.05% to about 0.50% of the composition. Preferably, diazolidinyl urea comprises from about 0.10% to about 0.30% of the composition. An optimum concentration of diazolidinyl urea is about 0.15% of the composition.

Allantoin can comprise from about 0.50% to about 10.0% of the composition. In one alternative, a preferred concentration of allantoin is from about 1.0% to about 2.0% of the composition. In this alternative, the optimum concentration of allantoin is about 1.50% of the composition. In another alternative, an optimum concentration of allantoin is about 9.00% of the composition.

If present, fragrance can comprise from about 0.05% to about 0.50% of the composition. Preferably, fragrance comprises from about 0.10% to about 0.40% of the composition. An optimum concentration of fragrance is about 0.20% of the composition.

Yet another embodiment of a method according to the present invention uses a composition comprising an oil-in-water emulsion comprising:

(1) allantoin; and

(2) an emulsifier system comprising:
(a) a polyethylene glycol ester of stearic acid; and
(b) glyceryl stearate.

Typically, the number of ethylene glycol moieties in the polyethylene glycol ester of stearic acid is from 25 to 100. Two preferred polyethylene glycol esters of stearic acid for use in compositions suitable for use in this embodiment of a method according to the present invention are PEG-40 stearate and PEG-100 stearate, with 40 and 100 ethylene glycol moieties respectively. A particularly preferred polyethylene glycol ester of stearic acid is PEG-100 stearate.

In this embodiment of a method according to the present invention, the composition further comprises an acid to adjust the pH to a value in a range of from about 3.0 to about 6.0. Preferably, the pH of the composition is from about 5.0 to about 5.8. The acid can be an inorganic or an organic acid as described above. Preferably, the acid is a weak organic acid. Most preferably, the acid is citric acid.

The composition can further comprise other ingredients. For example, the composition can further comprise a solvent component. Typically, the solvent component includes at least one solvent selected from the group consisting of ethylene glycol, propylene glycol, glycerin, and butylene glycol. Preferably, the solvent component is propylene glycol.

The composition can further comprise a chelator component. Preferably, the chelator component is tetrasodium ethylenediaminetetraacetic acid.

The composition can further comprise an emollient component. The emollient component can comprise at least one emollient selected from the group consisting of lanolin oil, cetyl alcohol, stearyl alcohol, and cod liver oil. Preferably, the emollient component comprises all of lanolin oil, cetyl alcohol, stearyl alcohol, and cod liver oil.

The composition can also further comprise an antioxidant. A preferred antioxidant is butylated hydroxytoluene. Other antioxidants such as butylated hydroxyanisole (BHA) can be used, alternatively or in addition to BHT.

The composition can further comprise a preservative component. The preservative component can comprise at least one preservative selected from the group consisting of methylparaben, propylparaben, and diazolidinyl urea. Preferably, the preservative component comprises all of methylparaben, propylparaben, and diazolidinyl urea.

The composition can further include fragrance as described above. The stability and function of the cream is not altered by the presence or absence of fragrance. As indicated above, it may be desirable to omit fragrance in some cases.

The following discussion describes ranges, preferred concentrations and optimum concentrations for preferred compositions with a pH of from about 5.0 to about 5.8 useful in methods of this embodiment of the present invention.

Water can comprise from about 50.0% to about 90.0% of the composition. Preferably, water comprises from about 55.0% to about 80.0% of the composition. In one alternative, in which the optimum concentration of allantoin is about 1.50% of the composition, the optimum concentration of water is about 67.86% of the composition. In another alternative, in which the optimum concentration of allantoin is about 9.00% of the composition, the optimum concentration of water is about 60.36% of the composition.

Propylene glycol can comprise from about 2.00% to about 9.00% of the composition. Preferably, propylene glycol comprises from about 4.30% to about 7.00% of the composition. An optimum concentration of propylene glycol is about 5.70% of the composition.

Tetrasodium EDTA can comprise from about 0.05% to about 0.50% of the composition. Preferably, PEG-100 stearate comprises from about 1.50% to about 3.00% of the composition. An optimum concentration of PEG-100 stearate is about 2.60% of the composition.

Lanolin oil can comprise from about 5.0% to about 15.0% of the composition. Preferably, lanolin oil comprises from about 8.0% to about 12.0% of the composition. An optimum concentration of lanolin oil is about 10.60% of the composition.

Cetyl alcohol can comprise from about 2.0% to about 10.0% of the composition. Preferably, cetyl alcohol comprises from about 2.50% to about 7.50% of the composition. An optimum concentration of cetyl alcohol is about 3.00% of the composition.

Stearyl alcohol can comprise from about 1.0% to about 4.0% of the composition. Preferably, stearyl alcohol comprises from about 1.0% to about 3.5% of the composition. An optimum concentration of stearyl alcohol is about 2.50% of the composition.

Glyceryl stearate can comprise from about 1.0% to about 5.0% of the composition. Preferably, glyceryl stearate comprises from about 2.0% to about 4.0% of the composition. An optimum concentration of glyceryl stearate is about 2.50% of the composition.

Cod liver oil can comprise from about 1.0% to about 7.0% of the composition. Preferably, cod liver oil comprises from about 1.0% to about 4.0% of the composition. An optimum concentration of cod liver oil is about 2.00% of the composition.

Butylated hydroxytoluene can comprise from about 0.10% to about 1.0% of the composition. Preferably, butylated hydroxytoluene comprises from about 0.20% to about 0.80% of the composition. An optimum concentration of butylated hydroxytoluene is about 0.50% of the composition.

Methylparaben can comprise from about 0.10% to about 0.50% of the composition. Preferably, methylparaben comprises from about 0.15% to 0.40% of the composition. An optimum concentration of methylparaben is about 0.30% of the composition.

Propylparaben can comprise from about 0.10% to about 0.50% of the composition. Preferably, propylparaben comprises from about 0.15% to about 0.40% of the composition. An optimum concentration of propylparaben is about 0.25% of the composition.

Diazolidinyl urea can comprise from about 0.05% to about 0.50% of the composition. Preferably, diazolidinyl urea comprises from about 0.10% to about 0.30% of the composition. An optimum concentration of diazolidinyl urea is about 0.20% of the composition.

Allantoin can comprise from about 0.50% to about 10.0% of the composition. In one alternative, a preferred concentration of allantoin is from about 1.0% to about 2.0% of the composition. In this alternative, the optimum concentration of allantoin is about 1.50% of the composition. In another alternative, an optimum concentration of allantoin is about 9.00% of the composition.

If present, fragrance can comprise from about 0.05% to about 0.50% of the composition. Preferably, fragrance comprises from about 0.10% to about 0.40% of the composition. An optimum concentration of fragrance is about 0.20% of the composition.

Yet another embodiment of a method according to the present invention employs a composition comprising an oil-in-water emulsion comprising:

(1) allantoin;

(2) a carbohydrate polymer; and

(3) an emulsifier system comprising:
(a) an acidic wax; and
(b) an anionic emulsifier that is substantially hydrophilic and is soluble in water.

Acidic waxes are those waxes having acidic groups that can be neutralized with alkaline materials such as hydroxides, alkoxides, unprotonated amines, and/or salts of strong bases and weak acids, such as sodium acetate. Upon neutralization, such waxes can act as emulsifiers or coemulsifiers. Particularly preferred acidic waxes include beeswax, carnauba wax, candelilla wax, siliconyl beeswax, siliconyl carnauba wax, and synthetic acidic waxes. Examples of synthetic acidic waxes are syncrowaxes marketed by Croda, Inc.

The carbohydrate polymer is typically selected from the group consisting of galactoarabinan, polygalactose, and polyarabinose. Preferably, the carbohydrate polymer is galactoarabinan.

The anionic emulsifier that is substantially hydrophilic and soluble in water can be selected from the group consisting of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, and sodium lauryl sulfate. A particularly preferred anionic emulsifier is sodium lauryl sulfate.

The pH of the composition is adjusted to a value in a range of between about 3.0 and about 6.0, typically with an acid. Preferably, the pH of the composition is from about 5.0 to about 6.0. The acid can be an inorganic or an organic acid as described above. Preferably, the acid is a weak organic acid. Most preferably, the acid is citric acid.

The composition used in this embodiment of a method according to the present invention can further comprise other ingredients. For example, the composition can further comprise a solvent component. Typically, the solvent component comprises at least one solvent selected from the group consisting of ethylene glycol, propylene glycol, glycerin, and butylene glycol. Preferably, the solvent component is propylene glycol.

The composition can further comprise a chelator component. Preferably, the chelator component is tetrasodium ethylenediaminetetraacetic acid.

The composition can further comprise an emollient component. The emollient component can comprise at least one emollient selected from the group consisting of lanolin oil, cetyl alcohol, stearyl alcohol, and cod liver oil. Preferably, the emollient component comprises all of lanolin oil, cetyl alcohol, stearyl alcohol, and cod liver oil

The composition can also further comprise an antioxidant. A preferred antioxidant is butylated hydroxytoluene. Other antioxidants such as butylated hydroxyanisole (BHA) can be used, alternatively or in addition to BHT.

The composition can further comprise a preservative component. The preservative component can comprise at least one preservative selected from the group consisting of methylparaben or propylparaben. Preferably, the preservative component comprises methylparaben and propylparaben.

The composition can further include fragrance as described above. The stability and function of the cream is not altered by the presence or absence of fragrance. As indicated above, it may be desirable to omit fragrance in some cases.

The following discussion describes ranges, preferred concentrations and optimum concentrations for preferred compositions with a pH of from about 5.0 to about 6.0 according to this embodiment of a method according to the present invention.

Water can comprise from about 50.0% to about 90.0% of the composition. Preferably, water comprises from about 52.0% to about 80% of the composition. In one alternative, in which the optimum concentration of allantoin is about 1.50% of the composition, the optimum concentration of water is about 61.65% of the composition. In another alternative, in which the optimum concentration of allantoin is about 9.00% of the composition, the optimum concentration of water is about 54.15% of the composition.

Propylene glycol can comprise from about 2.0% to about 9.0% of the composition. Preferably, propylene glycol comprises from about 4.0% to about 7.0% of the composition. An optimum concentration of propylene glycol is about 5.70% of the composition.

Sodium lauryl sulfate, as a 30% solution, can comprise from about 0.50% to about 5.0% of the composition. Preferably, sodium lauryl sulfate, as a 30% solution, comprises from about 1.0% to about 3.0% of the composition. An optimum concentration of sodium lauryl sulfate, as a 30% solution, is about 1.90% of the composition.

Tetrasodium EDTA can comprise from about 0.05% to about 0.30% of the composition. Preferably, tetrasodium EDTA comprises from about 0.10% to about 0.20% of the composition. An optimum concentration of tetrasodium EDTA is about 0.15% of the composition.

Galactoarabinan can comprise from about 1.0% to about 25.0% of the composition. Preferably, galactoarabinan comprises from about 3.0% to about 15.0% of the composition. An optimum concentration of galactoarabinan is about 5.00% of the composition.

Citric acid can comprise from about 0.05% to about 0.25% of the composition. Preferably, citric acid comprises from about 0.10% to about 0.20% of the composition. An optimum concentration of citric acid is about 0.15% of the composition.

Lanolin oil can comprise from about 5.0% to about 15.0% of the composition. Preferably, lanolin oil comprises from about 8.0% to about 12.0% of the composition. An optimum concentration of lanolin oil is about 10.60% of the composition.

Cetyl alcohol can comprise from about 1.0% to about 8.0% of the composition. Preferably, cetyl alcohol comprises from about 2.0% to about 7.0% of the composition. An optimum concentration of cetyl alcohol is about 4.20% of the composition.

Stearyl alcohol can comprise from about 0.50% to about 6.0% of the composition. Preferably, stearyl alcohol comprises from about 1.0% to about 4.0% of the composition. An optimum concentration of stearyl alcohol is about 2.00% of the composition.

An acidic wax such as beeswax can comprise from about 0.50% to about 5.0% of the composition. Preferably, the acidic wax such as beeswax comprises from about 1.0% to about 3.0% of the composition. An optimum concentration of the acidic wax such as beeswax is about 1.90% of the composition.

Cod liver oil can comprise from about 0.50% to about 15.0% of the composition. Preferably, cod liver oil comprises from about 1.0% to about 10.0% of the composition. An optimum concentration of cod liver oil is about 2.00% of the composition.

Butylated hydroxytoluene can comprise from about 0.1% to about 3.0% of the composition. Preferably, butylated hydroxytoluene comprises from about 0.25% to about 2.50% of the composition. An optimum concentration of butylated hydroxytoluene is about 0.50% of the composition.

Methylparaben can comprise from about 0.10% to about 0.50% of the composition. Preferably, methylparaben comprises from about 0.15% to about 0.40% of the composition. An optimum concentration of methylparaben is about 0.30% of the composition.

Propylparaben can comprise from about 0.10% to about 0.50% of the composition. Preferably, propylparaben comprises from about 0.15% to about 0.40% of the composition. An optimum concentration of propylparaben is about 0.25% of the composition.

Allantoin can comprise from about 0.50% to about 10.0% of the composition. In one alternative, a preferred concentration of allantoin is from about 1.0% to about 2.0% of the composition. In this alternative, the optimum concentration of allantoin is about 1.50% of the composition. In another alternative, an optimum concentration of allantoin is about 9.00% of the composition.

If present, fragrance can comprise from about 0.05% to about 0.50% of the composition. Preferably, if present, fragrance can comprise from about 0.10% to about 0.40% of the composition. An optimum concentration of fragrance is about 0.20% of the composition.

Yet another embodiment of a method according to the present invention employs a composition according to the present invention is a composition comprising an oil-in-water emulsion comprising:

(1) allantoin in a concentration of at least about 2.5%;

(2) an emulsifier system comprising:
(a) an acidic wax; and
(b) an anionic emulsifier that is substantially hydrophilic and is soluble in water.

The acidic waxes used are as described above. A particularly preferred acidic wax is beeswax.

The anionic emulsifier that is substantially hydrophilic and soluble in water can be selected from the group consisting of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, and sodium lauryl sulfate. A particularly preferred anionic emulsifier is sodium lauryl sulfate.

The pH of the composition is adjusted to a range of between about 3.0 and about 6.0, typically with an acid. The acid can be an inorganic or an organic acid as described above. Preferably, the acid is a weak organic acid. Most preferably, the acid is citric acid.

This embodiment can further comprise other ingredients. For example, this embodiment of the invention can further comprise a solvent component. Typically, the solvent component comprises at least one solvent selected from the group consisting of ethylene glycol, propylene glycol, glycerin, or butylene glycol. Preferably, the solvent component is propylene glycol.

This embodiment of the invention can further comprise a chelator component. Preferably, the chelator component is tetrasodium ethylenediaminetetraacetic acid.

This embodiment of the invention can further comprise an emollient component. The emollient component can comprise at least one emollient selected from the group consisting of lanolin oil, cetyl alcohol, and stearyl alcohol. Preferably, the emollient component comprises all of lanolin oil, cetyl alcohol, and stearyl alcohol.

This embodiment of the invention can further comprise a preservative component. The preservative component can comprise one or more of methylparaben or propylparaben. Preferably, the preservative component comprises methylparaben and propylparaben. As indicated above, other preservatives can also be used.

This embodiment of the composition can further include fragrance as described above. The stability and function of the cream are not altered by the presence or absence of fragrance. As indicated above, it may be desirable to omit fragrance in some cases.

The following discussion describes ranges, preferred concentrations and optimum concentrations for preferred compositions according to this embodiment of the present invention when the pH is adjusted to a range of from about 5.0 to about 6.0.

Water can comprise from about 50.0% to about 90.0% of this embodiment of the composition. An optimum concentration of water is about 58.98% of this embodiment of the composition

Propylene glycol can comprise from about 2.0% to about 9.0% of this embodiment of the composition. An optimum concentration of propylene glycol is about 5.70% of this embodiment of the composition.

Sodium lauryl sulfate, as a 30% solution, can comprise from about 0.50% to about 5.0% of this embodiment of the composition. An optimum concentration of sodium lauryl sulfate, as a 30% solution, is about 3.00% of this embodiment of the composition.

Tetrasodium EDTA can comprise from about 0.05% to about 0.50% of this embodiment of the composition. An optimum concentration of tetrasodium EDTA is about 0.15% of this embodiment of the composition.

Citric acid can comprise from about 0.05% to about 0.50% of this embodiment of the composition. An optimum concentration of citric acid is about 0.12% of this embodiment of the composition.

Lanolin oil can comprise from about 5.0% to about 15.0% of this embodiment of the composition. An optimum concentration of lanolin oil is about 10.60% of this embodiment of the composition.

Cetyl alcohol can comprise from about 3.0% to about 10.0% of this embodiment of the composition. An optimum concentration of cetyl alcohol is about 4.20% of this embodiment of the composition.

Stearyl alcohol can comprise from about 1.0% to about 5.0% of this embodiment of the composition. An optimum concentration of stearyl alcohol is about 2.00% of this embodiment of the composition.

An acidic wax such as beeswax can comprise from about 0.50% to about 5.0% of this embodiment of the composition. An optimum concentration of the acidic wax such as beeswax is about 3.00% of this embodiment of the composition.

Methylparaben can comprise from about 0.10% to about 0.50% of this embodiment of the composition. An optimum concentration of methylparaben is about 0.30% of this embodiment of the composition.

Propylparaben can comprise from about 0.10% to about 0.50% of this embodiment of the composition. An optimum concentration of propylparaben is about 0.25% of this embodiment of the composition.

Allantoin can comprise from about 2.5% to about 10.0% of this embodiment of the composition. An optimum concentration of allantoin is about 9.00% of this embodiment of the composition.

If present, fragrance can comprise from about 0.05% to about 0.50% of this embodiment of the composition. An optimum concentration of fragrance is about 0.20% of this embodiment of the composition.

Examples of particularly preferred compositions useful in methods according to the present invention are described below.

Compositions useful in methods according to the present invention can contain other, optional ingredients. For example, compositions useful in methods according to the present invention can contain lipid-soluble components such as, but not limited to, caprylic/capric triglycerides; steareth-2; steareth-21; polyglyceryl-3 beeswax; a branched-carboxylic acid ester of a branched-chain alcohol selected from the group consisting of isononyl isononanoate, isodecyl isononanoate, isooctyl isononanoate, isononyl isooctanoate, isodecyl isooctanonoate, isooctyl isooctanoate, isononyl isodecanoate, isooctyl isodecanoate, and isodecyl isodecanoate; an acrylates/C₁₀-C₃₀ alkyl acrylates cross-polymer; methylgluceth-20; a glyceryl ester of a long-chain fatty acid selected from the group consisting of glyceryl monostearate, glyceryl monopalmitate, and glyceryl monoarachidate; hydrogenated vegetable oil; squalane; C₁₂-C₁₅ alkyl benzoates; di-C₁₂-C₁₅ alkyl fumarate; cholesterol; lanolin alcohol; octyldodecanol; isostearic acid; a branched-chain neopentanoate selected from the group consisting of octyldodecyl neopentanoate, heptyldodecyl neopentanoate, nonyldodecyl neopentanoate, octylundecyl neopentanoate, heptylundecyl neopentanoate, nonylundecyl neopentanoate, octyltridecyl neopentanoate, heptyltridecyl neopentanoate, and nonyltridecyl neopentanoate; an arachidyl ester of a short-chain carboxylic acid selected from the group consisting of arachidyl propionate, arachidyl acetate, arachidyl butyrate, and arachidyl isobutyrate; a long-chain fatty acid ester of a medium-chain alcohol selected from the group consisting of octyl palmitate, octyl myristate, octyl stearate, heptyl palmitate, heptyl myristate, heptyl stearate, nonyl palmitate, nonyl myristate, and nonyl stearate; jojoba oil; a myristyl ester of a long-chain fatty acid selected from the group consisting of myristyl myristate, myristyl laurate, and myristyl palmitate; bisabolol; hydrogenated jojoba oil; jojoba esters; methylgluceth-20 sesquistearate; PPG-14 butyl ether; PPG-15 stearyl ether; PPG-1-isoceteth-3-acetate; laureth-2-benzoate; diisostearyl dimer dilinoleate; a long-chain cis-monounsaturated fatty acid ester of a medium-chain alcohol; a medium-chain saturated carboxylic acid ester of a long-chain alcohol; hydrogenated soy glycerides; a long-chain fatty acid ester of cetyl alcohol selected from the group consisting of cetyl palmitate, cetyl stearate, and cetyl myristate; palm kernel oil; palm oil; and an arachidyl ester selected from the group consisting of arachidyl acetate, arachidyl propionate, arachidyl butyrate, and arachidyl isobutyrate.

In addition, the compositions useful in methods according to the present invention can further comprise other ingredients that are generally used in the cosmetic art and in the art of over-the-counter skin preparations. These ingredients include, but are not limited to: (1) other plant extracts, such as horsetail extract, horse chestnut extract, rose extract, or lavender extract; (2) a short-chain carboxylic acid ester of tocopherol selected from the group consisting of tocopheryl acetate, tocopheryl propionate, tocopheryl butyrate, and tocopheryl isobutyrate; (3) a long-chain fatty acid ester of ascorbic acid selected from the group consisting of ascorbyl myristate, ascorbyl palmitate, and ascorbyl stearate; (4) a long-chain fatty acid ester of retinol or a retinal derivative or analogue wherein the acyl moiety of the ester is selected from the group consisting of myristic acid, palmitic acid, and stearic acid; and (5), a sunscreen, which can be at least one compound selected from the group consisting of octyl methoxycinnamate, p-aminobenzoic acid, ethyl p-aminobenzoate, isobutyl p-aminobenzoate, glyceryl p-aminobenzoate, p-dimethylaminobenzoic acid, methyl anthranilate, menthyl anthranilate, phenyl anthranilate, benzyl anthranilate, phenylethyl anthranilate, linalyl anthranilate, terpinyl anthranilate, cyclohexenyl anthranilate, amyl salicylate, phenyl salicylate, benzyl salicylate, menthyl salicylate, glyceryl salicylate, dipropyleneglycol salicylate, methyl cinnamate, benzyl cinnamate, a-phenyl cinnamonitrile, butyl cinnamoylpyruvate, umbelliferone, methylacetoumbelliferone, esculetin, methylesculetin, daphnetin, esculin, daphnin, diphenylbutadiene, stilbene, dibenzalacetone, benzalacetophenone, sodium 2-naphthol-3,6-disulfonate, sodium 2-naphthol-6,8-disulfonate, dihydroxynaphthoic acid, salts of dihydroxynaphthoic acid, o-hydroxybiphenyldisulfonates, p-hydroxybiphenyldisulfonates, 7-hydroxycoumarin, 7-methylcoumarin, 3-phenylcoumarin, 2-acetyl-3-bromoindazole, phenylbenzoxazole, methylnaphthoxazole, arylbenzothiazoles, quinine bisulfate, quinine sulfate, quinine chloride, quinine oleate, quinine tannate, 8-hydroxyquinoline salts, 2-phenylquinoline, hydroxy-substituted benzophenones, methoxy-substituted benzophenones, uric acid, vilouric acid, tannic acid, tannic acid hexaethylether, hydroquinone, oxybenzone, sulisobenzone, dioxybenzone, benzoresorcinol, 2,2′,4,4′-tetrahydroxybenzophenone, 2,2′-dihydroxy-4,4′-dimethoxybenzophenone, octabenzone, 4-isopropyldibenzoylmethane, butylmethoxydibenzoylmethane, etocrylene, and 4-isopropyldibenzoylmethane.

Other ingredients can also optionally be included in compositions useful in methods according to the present invention, such as colorants, pigments, opacifiers, and the like.

The compositions useful in methods according to the present invention are prepared by standard mixing techniques, such as are conventional in the cosmetic art and in the art of over-the-counter drug formulation for blending lipid-soluble components and water-soluble components. These mixing techniques include both manual and mechanical mixing, and include homogenization mixing and sweep mixing. The mixing techniques to be used can be chosen by one of ordinary skill in the art based on variables such as the viscosity of the components to be mixed and the volume of those components, as well as the relative proportion of lipid-soluble and water-soluble ingredients. The compositions can be mixed in two or more batches, such as one batch containing lipid-soluble ingredients and another batch containing water-soluble ingredients, and the batches can then be mixed at the final stage of preparation. In some cases, if triethanolamine is used, it is added last, as otherwise it may tend to thicken the emulsion. Other preparation methods are known in the art.

The dosages of the allantoin-containing composition to be administered and the frequency of those dosages can be determined by one of ordinary skill in the art depending on the particular disease affecting the patient, the clinical severity of the disease, the age and weight of the patient, the exposure of the patient to conditions that may precipitate outbreaks of dermatological or systemic inflammatory conditions, the degree of exposure to environmental insults, other drugs being administered, the response of the patient, and other pharmacokinetic factors generally understood in the art, such as liver and kidney metabolism. The interrelationship of dosages for animals of various sizes and species and humans based on mg/m³ of surface area is described in E. J. Freireich et al., “Quantitative Comparison of Toxicity of Anticancer Agents in Mouse, Rat, Hamster, Dog, Monkey and Man,” Cancer Chemother. Rep. 50:219-244 (1966).

Adjustments in the dosage regimen can be made to optimize the therapeutic response. Doses can be divided and administered on a daily basis or the dose can be reduced proportionately depending upon the therapeutic situation.

The allantoin-containing composition can be administered from once per day up to at least five times per day depending on the severity of the disease, the total dosage to be administered, and the judgment of the treating physician. In some cases, the allantoin-containing composition need not be administered on a daily basis, but can be administered every other day, every third day, or on other such schedules. However, it is generally preferred to administer the allantoin-containing composition daily.

In methods according to the present invention, the allantoin-containing composition can be administered alone or with other conventional therapeutic agents in a therapeutically effective quantity. These other therapeutic agents can either be applied topically to the skin or can be administered systemically, such as orally, intravenously, or by other conventional routes as generally known in the art. These agents can include steroids, nonsteroidal anti-inflammatory agents, leukotriene antagonists, monoclonal antibodies, and other agents. Additional agents can be administered to promote healing in the form of conventional creams or emulsions.

The invention is illustrated by the following Examples. These Examples are for illustrative purposes only and are not intended to limit the invention.

### EXAMPLES

### Example 1

### Preparation of Skin Protectant Over-the-Counter Cream with pH of 7.4

### (Prior Art Example)

A skin protectant over-the-counter (OTC) cream was prepared in accordance with the formulation of Table 1.

TABLE 1
COMPOSITION OF ALLANTOIN-CONTAINING SKIN
CREAM WITH pH OF 7.4

| | | | |
|---|---|---|---|
| INGREDIENT | RANGE | PREFERRED | OPTIMUM |

Part A

| | | | |
|---|---|---|---|
| Water | 50.0-90.0 | 55.0-75.0 | 66.20 |
| Sodium Lauryl Sulfate | 0.50-2.50 | 1.00-2.50 | 1.90 |
| (30%) | | | |
| Propylene Glycol | 2.0-9.0 | 3.0-6.0 | 5.30 |
| Tetrasodium EDTA | 0.05-0.50 | 0.10-0.30 | 0.15 |

Part B

| | | | |
|---|---|---|---|
| Lanolin Oil | 5.0-15.0 | 8.0-12.0 | 10.60 |
| Cetyl Alcohol | 3.0-10.0 | 3.5-7.5 | 6.80 |
| Stearyl Alcohol | 1.0-5.0 | 1.0-3.0 | 2.00 |
| Beeswax | 0.50-2.50 | 1.0-2.5 | 1.90 |
| Cod Liver Oil | 1.0-7.0 | 1.0-4.0 | 2.00 |
| BHT | 0.10-1.00 | 0.20-0.80 | 0.50 |

Part C

| | | | |
|---|---|---|---|
| St. John's Wort Extract | 0.05-0.50 | 0.05-0.15 | 0.10 |
| Witch Hazel Extract | 0.05-0.50 | 0.05-0.15 | 0.10 |
| Chamomile Extract | 0.05-0.50 | 0.05-0.15 | 0.10 |
| Arnica Extract | 0.05-0.50 | 0.05-0.15 | 0.10 |
| Methylparaben | 0.10-0.50 | 0.15-0.40 | 0.30 |
| Propylparaben | 0.10-0.50 | 0.10-0.30 | 0.25 |
| Allantoin | 0.50-2.00 | 0.50-2.00 | 1.50 |
| Fragrance | 0.05-0.50 | 0.10-0.30 | 0.20 |

The Part A ingredients were combined and heated to 175° F. with mixing. The Part B ingredients were combined and heated to 175° F. with mixing. The Part B mixture was then added to the Part A mixture with mixing. The resulting mixture was then cooled to 120° F. with continued mixing. The Part C ingredients were then added with mixing. The final emulsion was allowed to cool with continued mixing. The resulting cream had a pH of 7.4. Samples of the cream prepared from Example 1 were used for accelerated aging stability studies and analyzed for their allantoin concentration after a period of time at 40° C. The results are shown in Table 2.

As can be seen from Table 2, the allantoin in the cream from Example 1 undergoes degradation and would not meet the specifications required for an OTC drug.

TABLE 2
STABILITY OF ALLANTOIN IN SKIN CREAM COMPOSITION
OF EXAMPLE 1 WITH STORAGE AT 40° C.

| Days at 40° C. | Weight % Allantoin |
|---|---|
| 0 | 1.5 |
| 30 | 1.4 |
| 60 | 1.3 |
| 90 | 1.2 |

### Example 2

### Preparation of a Cream Containing Allantoin with Lower pH

An OTC skin cream containing allantoin was prepared using the ingredients in Table 3 to provide a cream with a lower pH.

TABLE 3
COMPOSITION OF ALLANTOIN-CONTAINING
SKIN CREAM WITH pH OF 5.3

| | | | |
|---|---|---|---|
| INGREDIENT | RANGE | PREFERRED | OPTIMUM |

Part A

| | | | |
|---|---|---|---|
| Water | 50.0-90.0 | 55.0-75.0 | 68.68 |
| Sodium Lauryl Sulfate | 0.50-2.50 | 1.00-2.50 | 1.90 |
| (30%) | | | |
| Propylene Glycol | 2.0-9.0 | 3.0-6.0 | 5.30 |
| Tetrasodium EDTA | 0.05-0.50 | 0.10-0.30 | 0.15 |
| Citric Acid | 0.05-0.50 | 0.08-0.35 | 0.12 |

Part B

| | | | |
|---|---|---|---|
| Lanolin Oil | 5.0-15.0 | 8.0-12.0 | 10.60 |
| Cetyl Alcohol | 3.0-10.0 | 3.5-7.5 | 4.20 |
| Stearyl Alcohol | 1.0-5.0 | 1.0-3.0 | 2.00 |
| Beeswax | 0.50-2.50 | 1.0-2.5 | 1.90 |
| Cod Liver Oil | 1.0-7.0 | 1.0-4.0 | 2.00 |
| BHT | 0.10-1.00 | 0.20-0.80 | 0.50 |

Part C

| | | | |
|---|---|---|---|
| St. John's Wort Extract | 0.05-0.50 | 0.05-0.15 | 0.10 |
| Witch Hazel Extract | 0.05-0.50 | 0.05-0.15 | 0.10 |
| Chamomile Extract | 0.05-0.50 | 0.05-0.15 | 0.10 |
| Arnica Extract | 0.05-0.50 | 0.05-0.15 | 0.10 |
| Methylparaben | 0.10-0.50 | 0.15-0.40 | 0.30 |
| Propylparaben | 0.10-0.50 | 0.10-0.30 | 0.25 |
| Allantoin | 0.50-10.00 | 0.50-2.00 | 1.50 |
| Fragrance | 0.05-0.50 | 0.10-0.30 | 0.20 |

The Part A ingredients were combined and heated to 175° F. with mixing. The Part B ingredients were combined and heated to 175° F. with mixing. The Part B mixture was added to the Part A mixture with mixing. The resulting mixture was then cooled to 120° F. with mixing at which time the Part C ingredients were added with mixing. The final emulsion was allowed to cool with continue mixing. The resulting cream had a pH of 5.3.

It was found that a similar cream was produced if Part B was added to Part A or Part A was added to Part B. However, the cream has a better appearance if the oil phase and water phase are homogenized under high shear after the two phases are added to one another.

Samples of the cream of this example were used for accelerated aging stability studies and analyzed for their allantoin concentration. The results are shown in Table 4. As can be seen from Table 4, the allantoin is stable over time in a cream with a pH of 5.3.

TABLE 4
STABILITY OF ALLANTOIN IN SKIN CREAM COMPOSITION
OF EXAMPLE 2 WITH STORAGE AT 40° C.

| Days at 40° C. | Weight % Allantoin |
|---|---|
| 0 | 1.4 |
| 30 | 1.4 |
| 60 | 1.4 |
| 90 | 1.4 |

### Example 3

### Preparation of Allantoin-Containing Skin Cream with Ionic Emulsifiers

An allantoin-containing skin cream with ionic emulsifiers is prepared according to Table 5. The preparation follows the method used in Example 2, with the ingredients in each of Part A, Part B, and Part C being combined separately and then Part B being added to Part A, with Part C then being added to the combination of Part A and Part B. The pH is adjusted to a value in a range of from about 5.0 to about 5.8 by neutralizing the stearic acid with enough triethanolamine to reach this pH. Other bases can be used instead of triethanolamine.

TABLE 5
ALLANTOIN-CONTAINING SKIN CREAM
WITH IONIC EMULSIFIERS

| | | | |
|---|---|---|---|
| INGREDIENT | RANGE | PREFERRED | OPTIMUM |

Part A

| | | | |
|---|---|---|---|
| Water | 50.0-90.0 | 60.0-85.0 | 71.70 |
| Propylene Glycol | 2.0-9.0 | 4.0-7.0 | 5.70 |
| Triethanolamine (99%) | 0.20-4.0 | 0.50-3.0 | 1.25 |

Part B

| | | | |
|---|---|---|---|
| Lanolin Oil | 5.0-15.0 | 8.0-12.0 | 10.60 |
| Cetyl Alcohol | 1.0-7.0 | 2.0-6.0 | 3.50 |
| Stearic Acid | 0.50-5.0 | 1.0-4.0 | 2.50 |
| Cod Liver Oil | 1.0-7.0 | 1.5-5.0 | 2.00 |
| Butylated Hydroxytoluene | 0.10-1.0 | 0.20-0.80 | 0.50 |

Part C

| | | | |
|---|---|---|---|
| Methylparaben | 0.10-0.50 | 0.15-0.40 | 0.30 |
| Propylparaben | 0.10-0.50 | 0.15-0.40 | 0.25 |
| Allantoin | 0.50-10.0 | 1.0-2.0 | 1.50 |
| Fragrance | 0.05-0.50 | 0.10-0.40 | 0.20 |

### Example 4

### Preparation of Allantoin-Containing Skin Cream with Lactylate Emulsifiers

An allantoin-containing skin cream with the emulsifiers sodium stearoyl lactylate and sodium isostearoyl lactylate is prepared according to Table 6. The preparation follows the method used in Example 3. The pH is adjusted by the addition of the appropriate quantity of citric acid.

TABLE 6
ALLANTOIN-CONTAINING SKIN CREAM
WITH LACTYLATE EMULSIFIERS

| INGREDIENT | RANGE | PREFERRED | OPTIMUM |
|---|---|---|---|
| Part A | | | |
| Water | 50.0-90.0 | 60.0-80.0 | 73.42 |
| Propylene Glycol | 2.0-9.0 | 4.0-7.0 | 5.70 |
| Citric Acid | 0.05-0.50 | 0.10-0.40 | 0.18 |
| Sodium Stearoyl Lactylate | 0.30-3.0 | 0.50-2.50 | 1.00 |
| Sodium Isostearoyl | 0.05-1.0 | 0.10-0.70 | 0.25 |
| Lactylate | 0.05-0.25 | 0.10-0.20 | 0.15 |
| Tetrasodium EDTA | | | |
| Part B | | | |
| Lanolin Oil | 5.0-15.0 | 8.0-12.0 | 10.60 |
| Cetyl Alcohol | 1.0-8.0 | 2.0-7.0 | 3.80 |
| Cod Liver Oil | 1.0-7.0 | 1.0-4.0 | 2.00 |
| Butylated Hydroxytoluene | 0.10-1.0 | 0.20-0.80 | 0.50 |
| Part C | | | |
| Methylparaben | 0.10-0.50 | 0.15-0.40 | 0.30 |
| Propylparaben | 0.10-0.50 | 0.15-0.40 | 0.25 |
| Allantoin | 0.50-10.0 | 1.0-2.0 | 1.50 |
| Fragrance | 0.05-0.50 | 0.10-0.40 | 0.20 |

### Example 5

### Preparation of Allantoin-Containing Skin Cream with Carboxypolymethylene Polymer

An allantoin-containing skin cream with carboxypolymethylene polymer is prepared according to Table 7. The preparation follows the method used in Example 3, except that the triethanolamine (Part D) is added last, after the combining of Parts A, B, and C, to avoid thickening of the emulsion. The triethanolamine is added to adjust the pH.

TABLE 7
ALLANTOIN-CONTAINING SKIN CREAM WITH
CARBOXYPOLYMETHYLENE POLYMER

| INGREDIENT | RANGE | PREFERRED | OPTIMUM |
|---|---|---|---|
| Part A | | | |
| Water | 50.0-90.0 | 60.0-80.0 | 73.55 |
| Carboxypolymethylene | 0.40-3.0 | 0.50-2.0 | 1.00 |
| Polymer | | | |
| Propylene Glycol | 2.0-9.0 | 4.0-7.0 | 5.70 |
| Part B | | | |
| Lanolin Oil | 5.0-15.0 | 8.0-12.0 | 10.00 |
| Cetyl Alcohol | 1.0-8.0 | 2.0-7.0 | 3.00 |
| Cod Liver Oil | 1.0-7.0 | 1.0-4.0 | 2.00 |
| Butylated Hydroxytoluene | 0.10-1.0 | 0.20-0.80 | 0.50 |
| Part C | | | |
| Methylparaben | 0.10-0.50 | 0.15-0.40 | 0.30 |
| Propylparaben | 0.10-0.50 | 0.15-0.40 | 0.25 |
| Allantoin | 0.50-10.0 | 1.0-2.0 | 1.50 |
| Fragrance | 0.05-0.50 | 0.10-0.40 | 0.20 |
| Part D | | | |
| Triethanolamine (99%) | 0.05-3.0 | 0.20-2.0 | 0.80 |

### Example 6

### Preparation of Allantoin-Containing Skin Cream with Polyethylene Glycol Ethers of Cetearyl Alcohol

An allantoin-containing skin cream with polyethylene glycol ethers of cetearyl alcohol is prepared according to Table 8. The preparation follows the method used in Example 3. The citric acid is added to adjust the pH.

TABLE 8
ALLANTOIN-CONTAINING SKIN CREAM WITH
POLYETHYLENE GLYCOL ETHERS OF CETEARYL ALCOHOL

| INGREDIENT | RANGE | PREFERRED | OPTIMUM |
|---|---|---|---|
| Part A | | | |
| Water | 50.0-90.0 | 55.0-75.0 | 66.33 |
| Propylene Glycol | 2.0-9.0 | 4.0-7.0 | 5.70 |
| Tetrasodium EDTA | 0.05-0.50 | 0.10-0.30 | 0.15 |
| Ceteareth-25 | 0.50-4.0 | 2.00-3.50 | 2.60 |
| Citric Acid | 0.04-0.40 | 0.10-0.30 | 0.12 |
| Part B | | | |
| Lanolin Oil | 5.0-15.0 | 8.0-12.0 | 10.60 |
| Cetyl Alcohol | 3.0-10.0 | 3.5-7.5 | 4.30 |
| Stearyl Alcohol | 1.0-5.0 | 2.0-4.0 | 3.50 |
| Ceteareth-6 | 0.50-4.0 | 1.0-3.0 | 1.80 |
| Cod Liver Oil | 1.0-7.0 | 1.0-4.0 | 2.00 |
| Butylated Hydroxytoluene | 0.10-1.0 | 0.20-0.80 | 0.50 |
| Part C | | | |
| Methylparaben | 0.10-0.50 | 0.15-0.40 | 0.30 |
| Propylparaben | 0.10-0.50 | 0.15-0.40 | 0.25 |
| Diazolidinyl Urea | 0.05-0.50 | 0.10-0.30 | 0.15 |
| Allantoin | 0.50-10.0 | 1.0-2.0 | 1.50 |
| Fragrance | 0.05-0.50 | 0.10-0.30 | 0.20 |

### Example 7

### Preparation of Allantoin-Containing Skin Cream with Polyethylene Glycol Ester of Stearic Acid and Glyceryl Stearate

An allantoin-containing skin cream with a polyethylene glycol ester of stearic acid and glyceryl stearate is prepared according to Table 9. The preparation follows the method used in Example 3. The citric acid is added to adjust the pH.

TABLE 9
ALLANTOIN-CONTAINING SKIN CREAM WITH POLYETHYLENE
GLYCOL ESTER OF STEARIC ACID AND GLYCERYL STEARATE

| INGREDIENT | RANGE | PREFERRED | OPTIMUM |
|---|---|---|---|
| Part A | | | |
| Water | 50.0-90.0 | 55.0-80.0 | 67.86 |
| Propylene Glycol | 2.0-9.0 | 4.3-7.0 | 5.70 |
| Tetrasodium EDTA | 0.05-0.50 | 0.10-0.30 | 0.15 |
| Citric Acid | 0.04-0.40 | 0.10-0.30 | 0.14 |
| PEG-100 Stearate | 1.0-5.0 | 1.5-3.0 | 2.60 |
| Part B | | | |
| Lanolin Oil | 5.0-15.0 | 2.0-12.0 | 10.60 |
| Cetyl Alcohol | 3.0-10.0 | 2.5-7.5 | 3.0 |
| Stearyl Alcohol | 1.0-4.0 | 1.0-3.5 | 2.50 |
| Glyceryl Stearate | 1.0-5.0 | 2.0-4.0 | 2.50 |
| Cod Liver Oil | 1.0-7.0 | 1.0-4.0 | 2.00 |
| Butylated Hydroxytoluene | 0.10-1.0 | 0.20-0.80 | 0.50 |
| Part C | | | |
| Methylparaben | 0.10-0.50 | 0.15-0.40 | 0.30 |
| Propylparaben | 0.10-0.50 | 0.15-0.40 | 0.25 |
| Diazolidinyl Urea | 0.05-0.50 | 0.10-0.30 | 0.20 |
| Allantoin | 0.50-10.0 | 1.0-2.0 | 1.50 |
| Fragrance | 0.05-0.50 | 0.10-0.40 | 0.20 |

### Example 8

### Preparation of Allantoin-Containing Skin Cream with Carboxypolymethylene Polymer and Polyethylene Glycol Ester of Stearic Acid

An allantoin-containing skin cream with a carboxypolymethylene polymer and a polyethylene glycol ester of stearic acid is prepared according to Table 10. The preparation follows the method used in Example 5, with the triethanolamine (Part D) being added last. The triethanolamine is added to adjust the pH.

TABLE 10
ALLANTOIN-CONTAINING SKIN CREAM WITH A
CARBOXYPOLYMETHYLENE POLYMER AND A
POLYETHYLENE GLYCOL ESTER OF STEARIC ACID

| INGREDIENT | RANGE | PREFERRED | OPTIMUM |
|---|---|---|---|
| Part A | | | |
| Water | 50.0-90.0 | 60.0-85.0 | 69.95 |
| Carboxypolymethylene | 0.30-3.0 | 0.50-2.0 | 0.85 |
| Polymer | 2.0-9.0 | 4.0-7.0 | 5.70 |
| Propylene Glycol | 0.25-2.5 | 0.50-2.0 | 1.50 |
| PEG-100 Stearate | | | |
| Part B | | | |
| Lanolin Oil | 5.0-15.0 | 8.0-12.0 | 10.60 |
| Cetyl Alcohol | 1.0-8.0 | 2.0-7.0 | 4.20 |
| Stearyl Alcohol | 0.50-6.0 | 0.75-5.0 | 1.50 |
| Cod Liver Oil | 1.0-7.0 | 1.0-4.0 | 2.00 |
| Butylated Hydroxytoluene | 0.10-1.0 | 0.20-0.80 | 0.50 |
| Part C | | | |
| Methylparaben | 0.10-0.50 | 0.15-0.40 | 0.30 |
| Propylparaben | 0.10-0.50 | 0.15-0.40 | 0.25 |
| Diazolidinyl Urea | 0.05-0.25 | 0.10-0.20 | 0.15 |
| Allantoin | 0.50-10.0 | 1.0-2.0 | 1.50 |
| Fragrance | 0.05-0.50 | 0.10-0.40 | 0.20 |
| Part D | | | |
| Triethanolamine (99%) | 0.05-3.0 | 0.20-2.0 | 0.80 |

### Example 9

### Preparation of Allantoin-Containing Skin Cream with Galactoarabinan. Sodium Lauryl Sulfate and Beeswax

An allantoin-containing skin cream with galactoarabinan, sodium lauryl sulfate, and beeswax is prepared according to Table 11. The preparation follows the method used in Example 3. The citric acid is used to adjust the pH.

TABLE 11
ALLANTOIN-CONTAINING SKIN CREAM WITH
GALACTOARABINAN. SODIUM
LAURYL SULFATE, AND BEESWAX

| INGREDIENT | RANGE | PREFERRED | OPTIMUM |
|---|---|---|---|
| Part A | | | |
| Water | 50.0-90.0 | 60.0-80.0 | 61.65 |
| Propylene Glycol | 2.0-9.0 | 4.0-7.0 | 5.70 |
| Sodium Lauryl Sulfate (30%) | 0.50-5.0 | 1.0-3.0 | 1.90 |
| Tetrasodium EDTA | 0.05-0.30 | 0.10-0.20 | 0.15 |
| Galactoarabinan | 1.0-25.0 | 3.0-15.0 | 5.00 |
| Citric Acid | 0.05-0.25 | 0.10-0.20 | 0.15 |
| Part B | | | |
| Lanolin Oil | 5.0-15.0 | 8.0-12.0 | 10.60 |
| Cetyl Alcohol | 1.0-8.0 | 2.0-7.0 | 4.20 |
| Stearyl Alcohol | 0.50-6.0 | 1.0-4.0 | 2.00 |
| Beeswax | 0.50-5.0 | 1.0-3.0 | 1.90 |
| Cod Liver Oil | 0.50-15.0 | 1.0-10.0 | 2.00 |
| Butylated Hydroxytoluene | 0.10-3.0 | 0.25-2.5 | 0.50 |
| Part C | | | |
| Methylparaben | 0.10-0.50 | 0.15-0.40 | 0.30 |
| Propylparaben | 0.10-0.50 | 0.15-0.40 | 0.25 |
| Allantoin | 0.50-10.0 | 1.0-2.0 | 1.50 |
| Fragrance | 0.05-0.50 | 0.10-0.40 | 0.20 |

### Example 10

### Preparation of a Cream Containing Allantoin with pH of 5.3 with High Allantoin Concentration

An OTC skin cream containing allantoin is prepared using the ingredients in Table 12 to provide a cream with a lower pH with an allantoin concentration of about 9.00%. The skin cream is prepared according to the method of Example 2.

TABLE 12
COMPOSITION OF ALLANTOIN-CONTAINING SKIN CREAM
WITH pH OF 5.3 WITH HIGH ALLANTOIN CONCENTRATION

| INGREDIENT | RANGE | OPTIMUM |
|---|---|---|
| Part A | | |
| Water | 50.0-90.0 | 61.38 |
| Sodium Lauryl Sulfate (30%) | 0.50-2.50 | 1.90 |
| Propylene Glycol | 2.0-9.0 | 5.30 |
| Tetrasodium EDTA | 0.05-0.50 | 0.15 |
| Citric Acid | 0.05-0.50 | 0.12 |
| Part B | | |
| Lanolin Oil | 5.0-15.0 | 10.60 |
| Cetyl Alcohol | 3.0-10.0 | 4.20 |
| Stearyl Alcohol | 1.0-5.0 | 2.00 |
| Beeswax | 0.50-2.50 | 1.90 |
| Cod Liver Oil | 1.0-7.0 | 2.00 |
| BHT | 0.10-1.00 | 0.50 |
| Part C | | |
| St. John's Wort Extract | 0.05-0.50 | 0.10 |
| Witch Hazel Extract | 0.05-0.50 | 0.10 |
| Chamomile Extract | 0.05-0.50 | 0.10 |
| Arnica Extract | 0.05-0.50 | 0.10 |
| Methylparaben | 0.10-0.50 | 0.30 |
| Propylparaben | 0.10-0.50 | 0.25 |
| Allantoin | 0.50-10.00 | 9.00 |
| Fragrance | 0.05-0.50 | 0.20 |

### Example 11

### Preparation of Allantoin-Containing Skin Cream with Ionic Emulsifiers with High Allantoin Concentration

An OTC skin cream containing allantoin is prepared using the ingredients in Table 13 to provide a cream with an allantoin concentration of about 9.00% using ionic emulsifiers. The skin cream is prepared according to the method of Example 3. The pH is adjusted to a value in a range of from about 5.0 to about 5.8 by neutralizing the stearic acid with enough triethanolamine to reach this pH. Other bases can be used instead of triethanolamine.

TABLE 13
ALLANTOIN-CONTAINING SKIN CREAM WITH IONIC
EMULSIFIERS WITH HIGH ALLANTOIN CONCENTRATION

| INGREDIENT | RANGE | OPTIMUM |
|---|---|---|
| Part A | | |
| Water | 50.0-90.0 | 64.20 |
| Propylene Glycol | 2.0-9.0 | 5.70 |
| Triethanolamine (99%) | 0.20-4.0 | 1.25 |
| Part B | | |
| Lanolin Oil | 5.0-15.0 | 10.60 |
| Cetyl Alcohol | 1.0-7.0 | 3.50 |
| Stearic Acid | 0.50-5.0 | 2.50 |
| Cod Liver Oil | 1.0-7.0 | 2.00 |
| Butylated Hydroxytoluene | 0.10-1.0 | 0.50 |
| Part C | | |
| Methylparaben | 0.10-0.50 | 0.30 |
| Propylparaben | 0.10-0.50 | 0.25 |
| Allantoin | 0.50-10.0 | 9.00 |
| Fragrance | 0.05-0.50 | 0.20 |

### Example 12

### Preparation of a Cream Containing Allantoin with Lactylate Emulsifiers with High Allantoin Concentration

An OTC skin cream containing allantoin is prepared using the ingredients in Table 14 to provide a cream with an allantoin concentration of about 9.00% using lactylate emulsifiers. The skin cream is prepared according to the method of Example 4. The pH is adjusted by the addition of the appropriate quantity of citric acid.

TABLE 14
ALLANTOIN-CONTAINING SKIN CREAM WITH LACTYLATE
EMULSIFIERS WITH HIGHALLANTOIN CONCENTRATION

| INGREDIENT | RANGE | OPTIMUM |
|---|---|---|
| Part A | | |
| Water | 50.0-90.0 | 65.92 |
| Propylene Glycol | 2.0-9.0 | 5.70 |
| Citric Acid | 0.05-0.50 | 0.18 |
| Sodium Stearoyl Lactylate | 0.30-3.0 | 1.00 |
| Sodium Isostearoyl Lactylate | 0.05-1.0 | 0.25 |
| Tetrasodium EDTA | 0.05-0.25 | 0.15 |
| Part B | | |
| Lanolin Oil | 5.0-15.0 | 10.60 |
| Cetyl Alcohol | 1.0-8.0 | 3.80 |
| Cod Liver Oil | 1.0-7.0 | 2.00 |
| Butylated Hydroxytoluene | 0.10-1.0 | 0.50 |
| Part C | | |
| Methylparaben | 0.10-0.50 | 0.30 |
| Propylparaben | 0.10-0.50 | 0.25 |
| Allantoin | 0.50-1,00 | 9.00 |
| Fragrance | 0.05-0.50 | 0.20 |

### Example 13

### Preparation of a Cream Containing Allantoin with Carboxypolymethylene Polymer with High Allantoin Concentration

An OTC skin cream containing allantoin is prepared using the ingredients in Table 15 to provide a cream with an allantoin concentration of about 9.00% with a carboxypolymethylene polymer. The skin cream is prepared according to the method of Example 5. Triethanolamine is added to adjust the pH.

TABLE 15
ALLANTOIN-CONTAINING SKIN CREAM
WITH CARBOXYPOLYMETHYLENE
POLYMER WITH HIGH ALLANTOIN CONCENTRATION

| INGREDIENT | RANGE | OPTIMUM |
|---|---|---|
| Part A | | |
| Water | 50.0-90.0 | 66.05 |
| Carboxypolymethylene Polymer | 0.40-3.0 | 1.00 |
| Propylene Glycol | 2.0-9.0 | 5.70 |
| Part B | | |
| Lanolin Oil | 5.0-15.0 | 10.00 |
| Cetyl Alcohol | 1.0-8.0 | 3.00 |
| Cod Liver Oil | 1.0-7.0 | 2.00 |
| Butylated Hydroxytoluene | 0.10-1.0 | 0.50 |
| Part C | | |
| Methylparaben | 0.10-0.50 | 0.30 |
| Propylparaben | 0.10-0.50 | 0.25 |
| Allantoin | 0.50-10.0 | 9.00 |
| Fragrance | 0.05-0.50 | 0.20 |
| Part D | | |
| Triethanolamine (99%) | 0.05-3.0 | 0.80 |

### Example 14

### Preparation of a Cream Containing Allantoin with Polyethylene Glycol Ethers of Cetearyl Alcohol with High Allantoin Concentration

An OTC skin cream containing allantoin is prepared using the ingredients in Table 16 to provide a cream with an allantoin concentration of about 9.00% with polyethylene glycol ethers of cetearyl alcohol. The skin cream is prepared according to the method of Example 6. The citric acid is added to adjust the pH.

TABLE 16
ALLANTOIN-CONTAINING SKIN CREAM WITH
POLYETHYLENE GLYCOL ETHERS OF CETEARYL
ALCOHOL WITH HIGH ALLANTOIN CONCENTRATION

| INGREDIENT | RANGE | OPTIMUM |
|---|---|---|
| Part A | | |
| Water | 50.0-90.0 | 58.83 |
| Propylene Glycol | 2.0-9.0 | 5.70 |
| Tetrasodium EDTA | 0.05-0.50 | 0.15 |
| Ceteareth-25 | 0.50-4.0 | 2.60 |
| Citric Acid | 0.04-0.40 | 0.12 |
| Part B | | |
| Lanolin Oil | 5.0-15.0 | 10.60 |
| Cetyl Alcohol | 3.0-10.0 | 4.30 |
| Stearyl Alcohol | 1.0-5.0 | 3.50 |
| Ceteareth-6 | 0.50-4.0 | 1.80 |
| Cod Liver Oil | 1.0-7.0 | 2.00 |
| Butylated Hydroxytoluene | 0.10-1.0 | 0.50 |
| Part C | | |
| Methylparaben | 0.10-0.50 | 0.30 |
| Propylparaben | 0.10-0.50 | 0.25 |
| Diazolidinyl Urea | 0.05-0.50 | 0.15 |
| Allantoin | 0.50-10.0 | 9.00 |
| Fragrance | 0.05-0.50 | 0.20 |

### Example 15

### Preparation of a Cream Containing Allantoin with Polyethylene Glycol Ethers of Stearic Acid and Glyceryl Stearate with High Allantoin Concentration

An OTC skin cream containing allantoin is prepared using the ingredients in Table 17 to provide a cream with an allantoin concentration of about 9.00% with polyethylene glycol ethers of stearic acid and glyceryl stearate. The skin cream is prepared according to the method of Example 7. The citric acid is added to adjust the pH.

TABLE 17
ALLANTOIN-CONTAINING SKIN CREAM WITH POLYETHYLENE
GLYCOL ESTER OF STEARIC ACID AND GLYCERYL STEARATE
WITH HIGH ALLANTOIN CONCENTRATION

| INGREDIENT | RANGE | OPTIMUM |
|---|---|---|
| Part A | | |
| Water | 50.0-90.0 | 60.36 |
| Propylene Glycol | 2.0-9.0 | 5.70 |
| Tetrasodium EDTA | 0.05-0.50 | 0.15 |
| Citric Acid | 0.04-0.40 | 0.14 |
| PEG-100 Stearate | 1.0-5.0 | 2.60 |
| Part B | | |
| Lanolin Oil | 50-15.0 | 10.60 |
| Cetyl Alcohol | 3.0-10.0 | 4.30 |
| Stearyl Alcohol | 1.0-5.0 | 3.50 |
| Glyceryl Stearate | 1.0-5.0 | 2.50 |
| Cod Liver Oil | 1.0-7.0 | 2.00 |
| Butylated Hydroxytoluene | 0.10-1.0 | 0.50 |
| Part C | | |
| Methylparaben | 0.10-0.50 | 0.30 |
| Propylparaben | 0.10-0.50 | 0.25 |
| Diazolidinyl Urea | 0.05-0.50 | 0.20 |
| Allantoin | 0.50-10.0 | 9.00 |
| Fragrance | 0.05-0.50 | 0.20 |

### Example 16

### Preparation of a Cream Containing Allantoin with a Carboxypolymethylene Polymer and a Polyethylene Glycol Ether of Stearic Acid with High Allantoin Concentration

An OTC skin cream containing allantoin is prepared using the ingredients in Table 18 to provide a cream with an allantoin concentration of about 9.00% with a carboxypolymethylene polymer and a polyethylene glycol ester of stearic acid. The skin cream is prepared according to the method of Example 8. The triethanolamine is added to adjust the pH.

TABLE 18
ALLANTOIN-CONTAINING SKIN CREAM WITH A
CARBOXYPOLYMETHYLENE POLYMER AND A
POLYETHYLENE GLYCOL ESTER OF STEARIC ACID
WITH HIGH ALLANTOIN CONCENTRATION

| INGREDIENT | RANGE | OPTIMUM |
|---|---|---|
| Part A | | |
| Water | 50.0-90.0 | 62.45 |
| Carboxypolymethylene Polymer | 0.30-3.0 | 0.85 |
| Propylene Glycol | 2.0-9.0 | 5.70 |
| PEG-100 Stearate | 0.25-2.5 | 1.50 |
| Part B | | |
| Lanolin Oil | 5.0-15.0 | 10.60 |
| Cetyl Alcohol | 1.0-8.0 | 4.20 |
| Stearyl Alcohol | 0.50-6.0 | 1.50 |
| Cod Liver Oil | 1.0-7.0 | 2.00 |
| Butylated Hydroxytoluene | 0.10-1.0 | 0.50 |
| Part C | | |
| Methylparaben | 0.10-0.50 | 0.30 |
| Propylparaben | 0.10-0.50 | 0.25 |
| Diazolidinyl Urea | 0.05-0.25 | 0.15 |
| Allantoin | 0.50-9.0 | 9.00 |
| Fragrance | 0.05-0.50 | 0.20 |
| Part D | | |
| Triethanolamine (99%) | 0.05-3.0 | 0.80 |

### Example 17

### Preparation of a Cream Containing Allantoin with Galactoarabinan. Sodium Lauryl Sulfate, and Beeswax with High Allantoin Concentration

An OTC skin cream containing allantoin is prepared using the ingredients in Table 19 to provide a cream with an allantoin concentration of about 9.00% with galactoarabinan, sodium lauryl sulfate, and beeswax. The skin cream is prepared according to the method of Example 9. The citric acid is used to adjust the pH. Another acidic wax can substitute for beeswax.

TABLE 19
COMPOSITION OF ALLANTOIN-CONTAINING SKIN CREAM
WITH GALACTOARABINAN. SODIUM LAURYL SULFATE,
AND BEESWAX WITH HIGH ALLANTOIN CONCENTRATION

| INGREDIENT | RANGE | OPTIMUM |
|---|---|---|
| Part A | | |
| Water | 50.0-90.0 | 54.15 |
| Propylene Glycol | 2.0-9.0 | 5.70 |
| Sodium Lauryl Sulfate (30%) | 0.50-5.0 | 1.90 |
| Tetrasodium EDTA | 0.05-0.30 | 0.15 |
| Galactoarabinan | 1.0-25.0 | 5.00 |
| Citric Acid | 0.05-0.25 | 0.15 |
| Part B | | |
| Lanolin Oil | 5.0-15.0 | 10.60 |
| Cetyl Alcohol | 1.0-8.0 | 4.20 |
| Stearyl Alcohol | 0.50-6.0 | 2.00 |
| Beeswax | 0.50-5.0 | 1.90 |
| Cod Liver Oil | 0.50-15.0 | 2.00 |
| Butylated Hydroxytoluene | 0.10-3.0 | 0.50 |
| Part C | | |
| Methylparaben | 0.10-0.50 | 0.30 |
| Propylparaben | 0.10-0.50 | 0.25 |
| Allantoin | 0.50-10.0 | 9.00 |
| Fragrance | 0.05-0.50 | 0.20 |

### Example 18

### Preparation of a Cream Containing Allantoin with Sodium Lauryl Sulfate and Beeswax with High Allantoin Concentration and pH of 3.9

An OTC skin cream containing allantoin is prepared using the ingredients in Table 20 to provide a cream with an allantoin concentration of about 9.00% with a pH of 3.9 with sodium lauryl sulfate and beeswax. The water phase (Part A in Table 20) was heated to 160-180° F. The oil phase (Part B in Table 20) was heated to 160-180° F. The heated oil phase was added to the heated water phase with continuing mixing to form an oil-in-water emulsion when this system was cooled. Between 115-125° F., the ingredients in Part C of Table 20 were added to the emulsion under high shear mixing. The final 9.00% allantoin cream had a pH of 3.90 and excellent emulsion stability when analyzed after aging 6 months at 40° C. The top, middle, and bottom of the batch were analyzed for allantoin and was found to contain 9.65%, 9.57%, and 9.66% respectively. After standing in a jar for three months at room temperature, the top, middle, and bottom of the jar were analyzed and the allantoin concentration at each point was found to be 9.59%, 9.57, and 9.58% respectively, showing that the allantoin does not precipitate in the jar on standing after manufacture. Analysis of a sample of the 9% allantoin cream after aging at 40° C. for eight months yielded 9.87% allantoin. This is within the specification that the active be within ±10% of its initial level after aging. The slightly higher value of 9.87 may indicate that some water has been lost on aging.

TABLE 20
COMPOSITION OF ALLANTOIN-CONTAINING SKIN
CREAM WITH pH OF 3.9 WITH BEESWAX AND HIGH
ALLANTOIN CONCENTRATION

| INGREDIENT | RANGE | OPTIMUM |
|---|---|---|
| Part A | | |
| Water | 50.0-90.0 | 58.98 |
| Sodium Lauryl Sulfate (30%) | 0.50-5.0 | 3.00 |
| Propylene Glycol | 2.0-9.0 | 5.70 |
| Tetrasodium EDTA | 0.05-0.50 | 0.15 |
| Citric Acid | 0.05-0.50 | 0.12 |
| Part B | | |
| Lanolin Oil | 5.0-15.0 | 10.60 |
| Cetyl Alcohol | 3.0-10.0 | 4.20 |
| Stearyl Alcohol | 1.0-5.0 | 2.00 |
| Beeswax | 0.50-5.0 | 3.00 |
| Part C | | |
| Methylparaben | 0.10-0.50 | 0.30 |
| Propylparaben | 0.10-0.50 | 0.25 |
| Fragrance | 0.05-0.50 | 0.20 |
| Allantoin | 2.50-10.0 | 9.00 |

### Example 19

### Treatment of Epidermolysis Bullosa with Allantoin-Containing Skin Cream

A female epidermolysis bullosa patient (A.B.) was treated with the allantoin-containing skin cream of Example 2 prepared in accordance with the optimum formulation recited in Table 3. The allantoin-containing skin cream used for treatment comprised 68.68% water, 1.90% 30% sodium lauryl sulfate solution, 0.15% tetrasodium EDTA, 0.12% citric acid, 10.60% lanolin oil, 4.20% cetyl alcohol, 2.00% stearyl alcohol, 1.90% beeswax, 2.00% cod liver oil, 0.50% butylated hydroxytoluene, 0.10% St. John's wort, 0.10% chamomile extract, 0.10% witch hazel extract, 0.10% arnica extract, 0.30% methylparaben, 0.20% propylparaben, 1.50% allantoin, and 0.20% fragrance. The patient A.B. was born with recessive dystrophic epidermolysis bullosa. She was born with no skin on her right foot from the shin down and spent the first 32 days of her life in intensive care. Her skin, which was constantly covered with Aquaphor, had the strength of tissue paper and blistered from the slightest touch. Although her feet, legs, arms, and hands were bandaged constantly, they continued to blister beneath the bandages. Her daily dressing changes took over an hour, and she required pain medication prior to each dressing change. Regardless of the meticulous care that the patient received, she battled infection constantly and chronic areas refused to heal. She began to develop infections that her doctors were unable to treat with antibiotics. Since her birth, the patient had required several different topical and oral antibiotics, as well as intramuscular injections. Because of the poor condition of her feet, the occupational and physical therapists treating the patient seriously doubted that she would ever walk.

The skin cream of Example 2 began to be applied to the patient A.B. when she was approximately 9½ months old. The registered nurses that cared for the patient A.B. at her home immediately observed that the cream cut the healing time for an open wound in half and actually kept blisters from spreading over larger areas. As absolutely no irritation was observed and tremendous improvement was seen for the areas receiving the cream, the cream then was applied to all unbandaged areas of the body of the patient 5 to 6 times daily. A remarkable reduction in the number of blisters was noticed, and the purplish colors of the scars began to fade. After continued success with the skin cream of Example 2, it began to be used on the patient under the bandaged areas in place of the Aquaphor.

For approximately four months, the cream was applied to both the bandaged and the unbandaged areas of the patient A.B. For the first time since her birth, her right foot completely healed and was without any open sore or blister. The registered nurses that cared for the patient in her home continued to note a remarkable reduction in the amount of blistering, both under the bandages and on the open skin. The healing time for newly blistered areas was much faster. The areas healed without the milia cysts that, prior to using the cream, accompanied each scar.

No type of antibiotic has been applied to the patient since the cream started to be used on the patient. Despite the lack of use of antibiotic, her foot remained infection free. The period during which the cream of Example 2 was used was the longest period for which her foot had gone without reblistering and/or becoming infected.

At the last visit of the doctors to the patient, the doctors were amazed to see skin on areas of the foot that they never thought would heal. The patient is able to walk better and for longer areas of time, and she was able to actually run across the floor.

The patient had experienced an overall decrease in skin fragility. Her right lower extremity, the area of greatest blistering, has continued to have decreased erythema, decreased pain, and decreased skin fragility. The patient did not require any bacterial cultures or antibiotics over the period during which the skin cream of Example 2 was used.

Dressing changes were accomplished in half the time and without any pain medication. The mother of the patient was able to actually change her dressings alone. Before the cream of Example 2 was applied to the patient, this task was impossible for the mother of the patient because of the poor condition of her feet and the additional steps and time necessary to change the dressings prior to the use of the cream of Example 2 on the patient.

One area of the patient did not receive the cream: the buttocks area. At one point, the patient developed two very small blisters in that area about the size of a dime. One of the blisters continued to spread and spread until the blister covered the entire buttocks area. The area was raw and the blister continued to refill. No area on her body had had blisters spread since the cream of Example 2 had been used on the patient. This is the only area in which the cream was not used because no blisters had developed in that area prior to this. This strongly suggests that the cream was responsible for making a remarkable difference in the healing and protection of the skin of the patient.

Although the patient, as with all patients with recessive dystrophic epidermolysis bullosa, continued to have areas of scarring on hands with concern of eventual fusion and decreased function, her disease had stabilized after the use of the cream of Example 2.

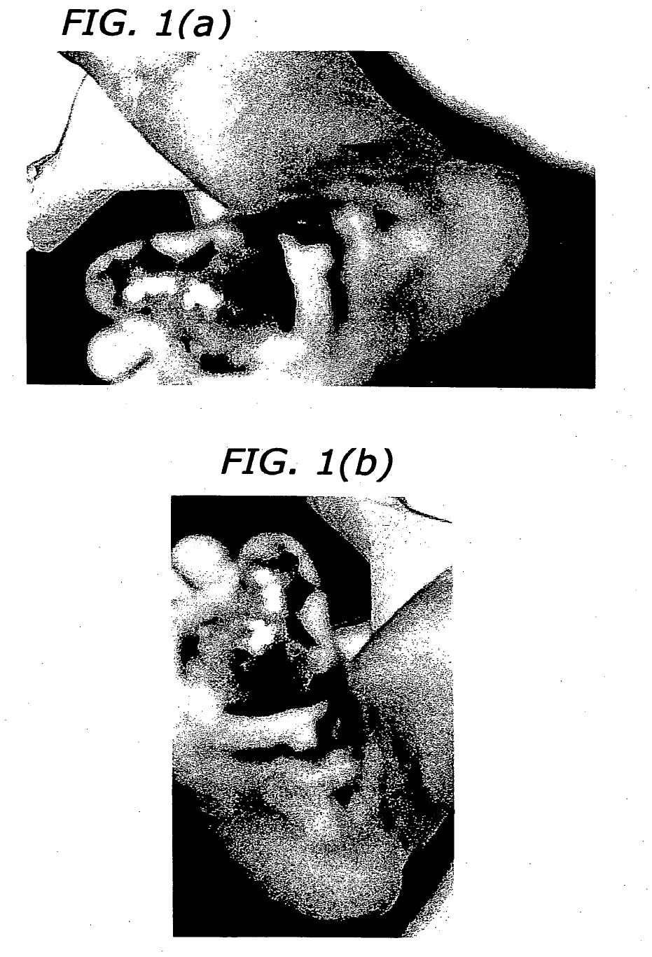

FIGS. 1(a) and 1(b) are pictures of the right foot of the patient A.B. before the use of the cream of Example 2 from two different views, showing the severity of the disease.

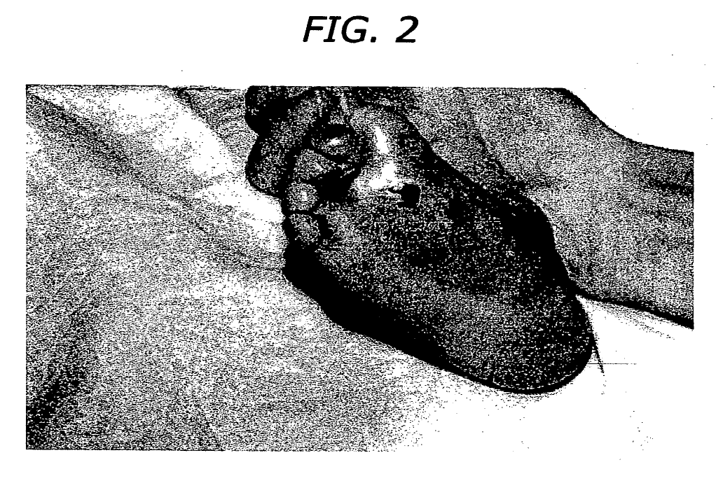

FIG. 2 is a picture of the right foot of the patient A.B. after two months of use of the cream of Example 2, showing considerable improvement.

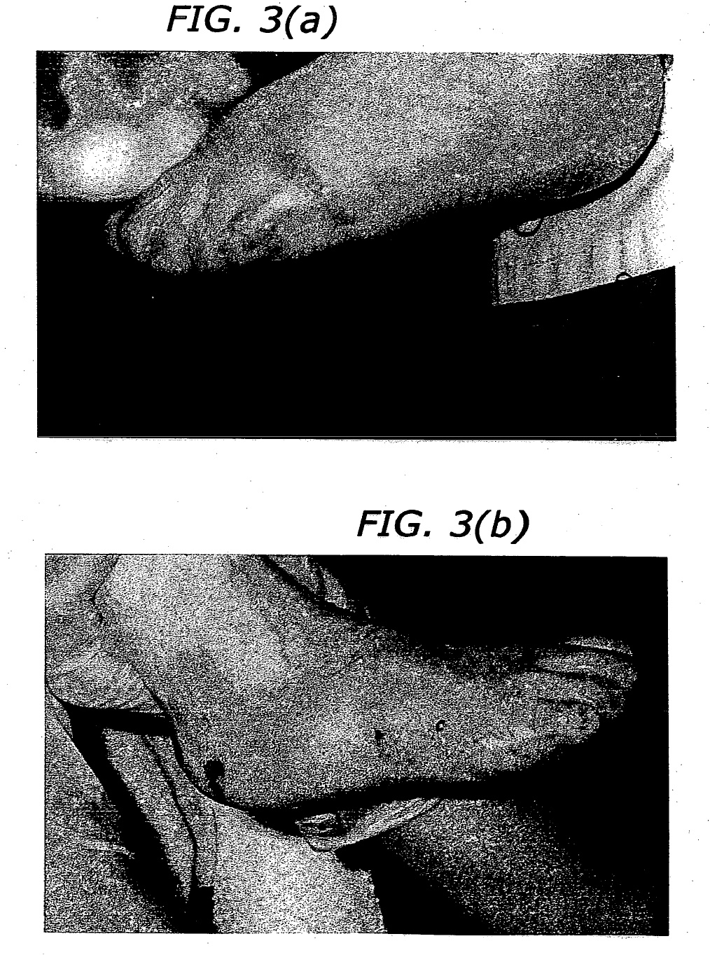

FIGS. 3(a) and 3(b) are pictures of the right foot of the patient A.B. after 12 months of use of the cream of Example 2, showing substantial improvement and clearing of the lesions.

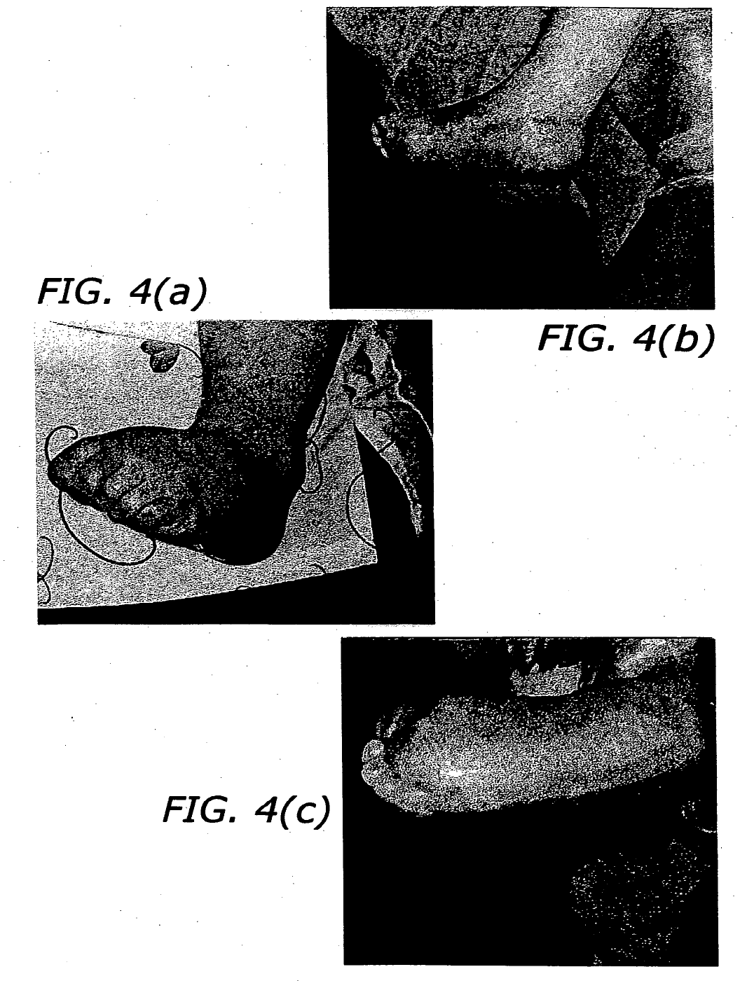

FIGS. 4(a), 4(b), and 4(c) are additional pictures of the right foot of the patient A.B. after 12 months of use of the cream of Example 2, again showing substantial improvement and clearing of the lesions.

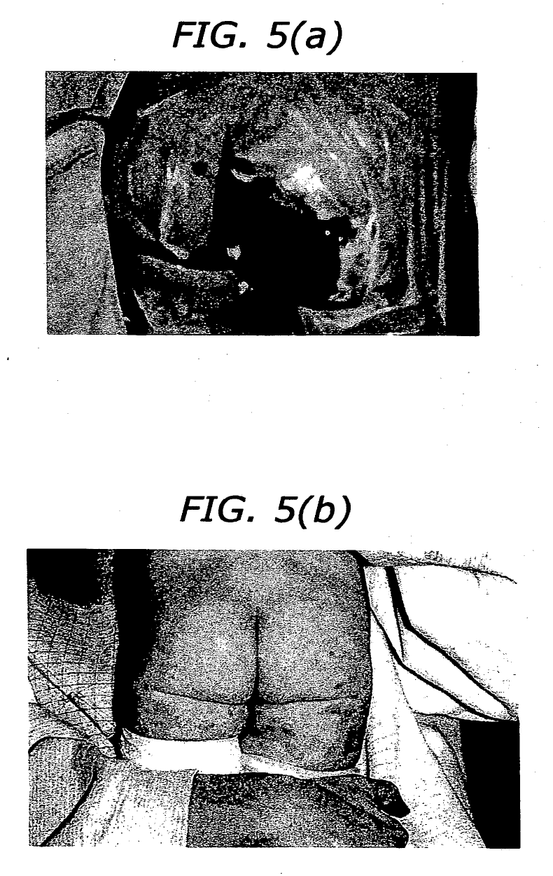

FIGS. 5(a) and 5(b) are pictures of the buttocks area of the patient A.B. before the use of the cream (FIG. 5(a)) and after 2 weeks of use of the cream (FIG. 5(b)), showing substantial improvement and clearing of the lesions.

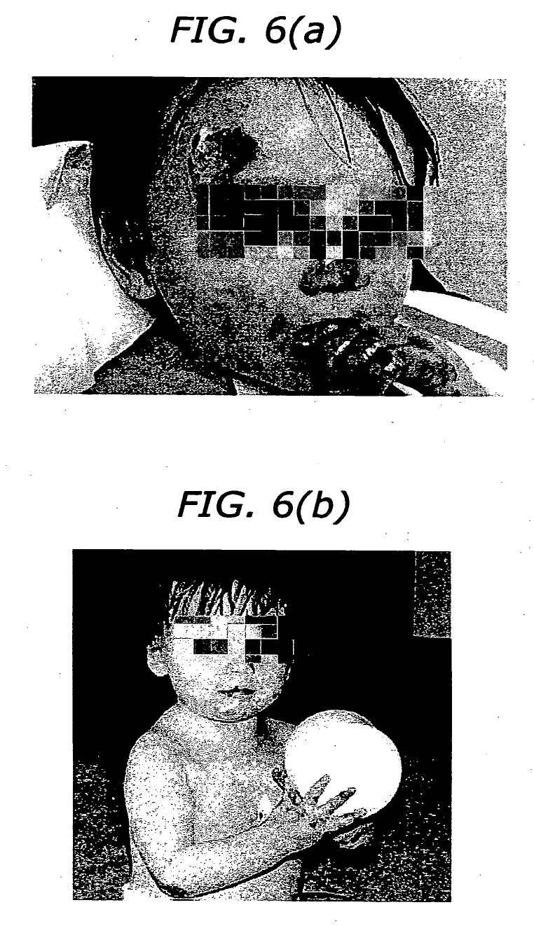

FIGS. 6(a) and 6(b) are pictures of the facial area of the patient A.B. before the use of the cream (FIG. 6(a)) and after 3 months of use of the cream (FIG. 6(b)), showing substantial improvement, fading, and clearing of the lesions.

### Example 18

### Preparation of a Cream Containing Allantoin with Sodium Lauryl Sulfate, and Beeswax with High Allantoin Concentration and pH of 3.9

An OTC skin cream containing allantoin is prepared using the ingredients in Table 20 to provide a cream with an allantoin concentration of about 9.00% with a pH of 3.9 with sodium lauryl sulfate and beeswax. The water phase (Part A in Table 20) was heated to 160-180° F. The oil phase (Part B in Table 20) was heated to 160-180° F. The heated oil phase was added to the heated water phase with continuing mixing to form an oil-in-water emulsion when this system was cooled. Between 115-125° F., the ingredients in Part C of Table 20 were added to the emulsion under high shear mixing. The final 9.00% allantoin cream had a pH of 3.90 and excellent emulsion stability when analyzed after aging 6 months at 40° C. The top, middle, and bottom of the batch were analyzed for allantoin and was found to contain 9.65%, 9.57%, and 9.66% respectively. After standing in a jar for three months at room temperature, the top, middle, and bottom of the jar were analyzed and the allantoin concentration at each point was found to be 9.59%, 9.57, and 9.58% respectively, showing that the allantoin does not precipitate in the jar on standing after manufacture. Analysis of a sample of the 9% allantoin cream after aging at 40° C. for eight months yielded 9.87% allantoin. This is within the specification that the active be within ±10% of its initial level after aging. The slightly higher value of 9.87 may indicate that some water has been lost on aging.

### Example 20

### Treatment of Epidermolysis Bullosa

A female epidermolysis bullosa patient (C.D.) was treated with the allantoin-containing skin cream of Example 2. The patient C.D. had epidermolysis bullosa of the Dowling-Meara type.

The patient C.D. received two to three applications per day of the allantoin-containing skin cream of Example 2. The cream produced considerable improvement in the skin of the patient C.D. This was the first time that her skin had remained moderately clear for a long period of time. The areas that experienced severe blistering had remained clean with the exception of some minor blistering. This blistering was not nearly as severe as what had been experienced prior to the use of the skin cream of Example 2. Also, a blister that did start on the back of the patient C.D. did not develop into a full-blown, spread-wide blister as had happened previously. This tendency of these blisters to spread is characteristic of the Dowling-Meara form of epidermolysis bullosa. Even problem areas that have taken a longer time to heal have not spread out of control.

The time required for the care of the patient C.D., such as the time required for lancing and wrapping her wounds, has decreased by at least 75% subsequent to the administration of the allantoin-containing skin cream of Example 2. The requirements for medical supplies used for the care of the patient C.D., such as sterile needles, sterile bandages, and sterile dressing sponges, also decreased tremendously subsequent to the administration of the allantoin-containing skin cream of Example 2.

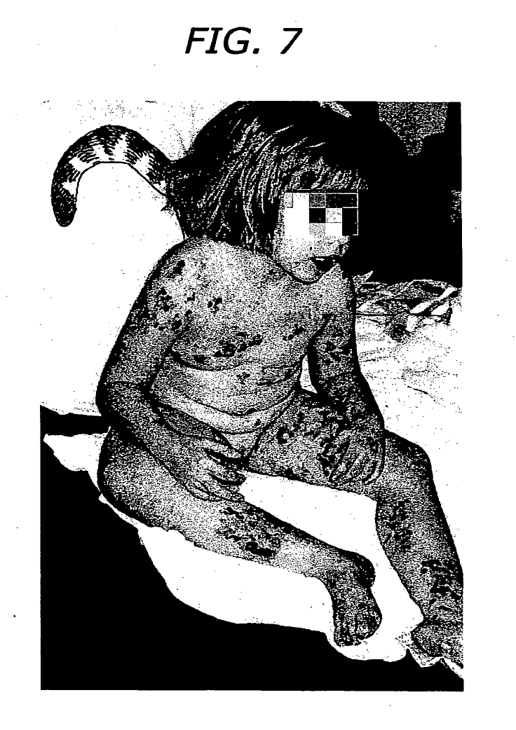

FIG. 7 is a photograph of patient C.D. before commencement of the use of the allantoin-containing skin cream of Example 2.

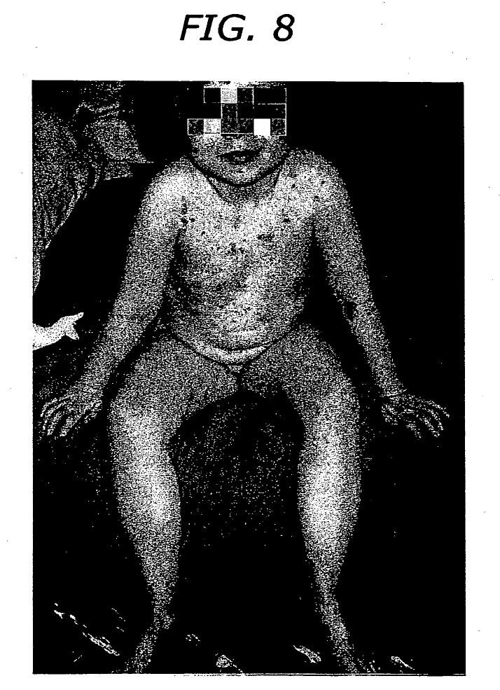

FIG. 8 is a photograph of patient C.D. after 8 weeks of use of the allantoin-containing skin cream of Example 2, showing substantial improvement of the lesions.

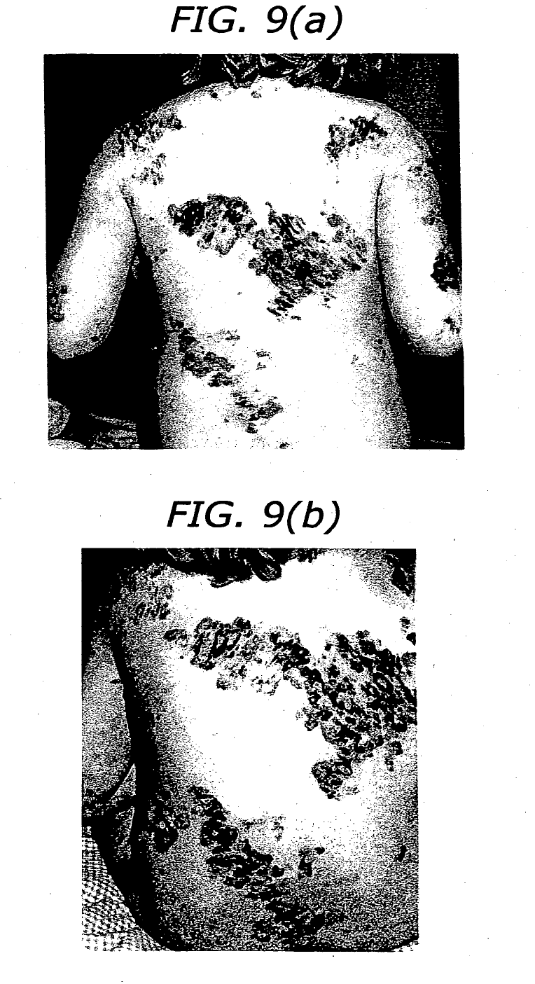

FIG. 9(a) is a photograph of the back area of patient C.D. before commencement w of the use of the allantoin-containing skin cream of Example 2.

FIG. 9(b) is another photograph of the back area of patient C.D. before commencement of the use of the allantoin-containing skin cream of Example 2.

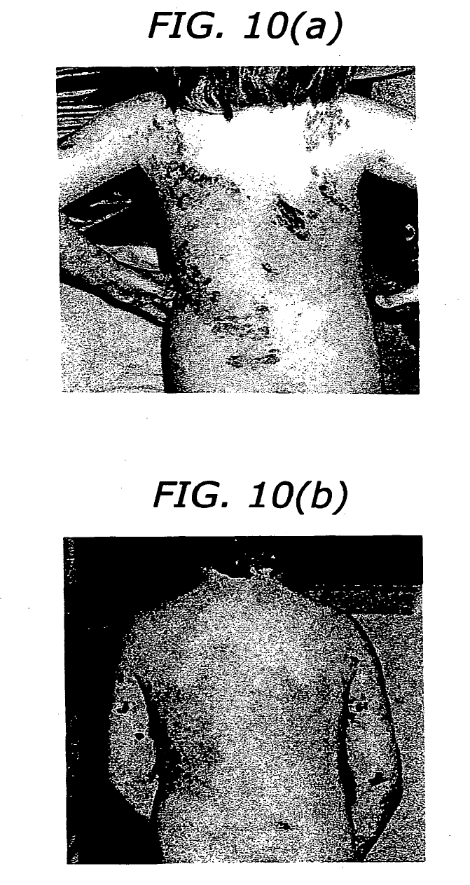

FIG. 10(a) is a photograph of the upper back area of patient C.D. after 2 weeks of use of the allantoin-containing skin cream of Example 2, showing considerable improvement.

FIG. 10(b) is a photograph of the upper back area of patient C.D. after 8 weeks of use of the allantoin-containing skin cream of Example 2, showing continued improvement evidenced by fading of the lesions.

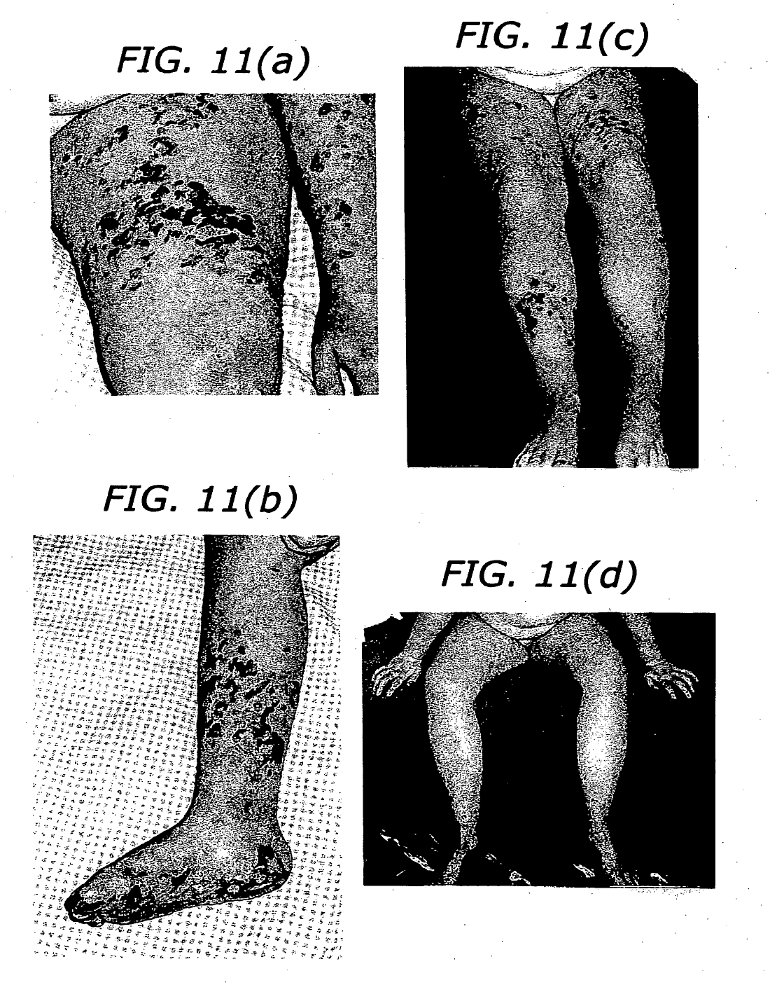

FIG. 11(a) is a photograph of the upper leg area of patient C.D. before commencement of the use of the allantoin-containing skin cream of Example 2.

FIG. 11(b) is a photograph of the lower leg area of patient C.D. before commencement of the use of the allantoin-containing skin cream of Example 2.

FIG. 11(c) is a photograph of the legs of patient C.D. after 2 weeks of use of the allantoin-containing skin cream of Example 2, showing substantial improvement.

FIG. 11(d) is a photograph of the legs of patient C.D. after 8 weeks of use of the allantoin-containing skin cream of Example 2, showing continuing improvement.

### ADVANTAGES OF THE PRESENT INVENTION

The present invention provides an improved method of treating skin diseases and conditions characterized by ulceration, inflammation, and blistering. This includes such difficult-to-treat conditions as epidermolysis bullosa, decubitus ulcers, diabetic ulcers, pressure ulcers, and milia, as well as other inflammatory conditions. Methods according to the present invention provide rapid improvement, are well tolerated by patients, are easy to apply, and can be used alone or with other methods for treatment of skin conditions.

Although the present invention has been described in considerable detail, with reference to certain preferred versions thereof, other versions and embodiments are possible. Therefore, the scope of the invention is determined by the following claims.

While the specification describes particular embodiments of the present invention, those of ordinary skill can devise variations of the present invention without departing from the inventive concept.

1-173. (canceled)

174. A method of treating a skin condition or disease characterized by ulceration, inflammation, or blistering of the skin comprising applying to the skin an allantoin-containing composition in a therapeutically effective amount, wherein the allantoin-containing composition comprises an oil-in-water emulsion comprising:
greater than 2.0% allantoin;
an emulsifier system including:
(i) an acidic wax; and
(ii) an anionic emulsifier that is substantially hydrophilic and is soluble in water; and an acid to adjust the pH of the composition to a value in the range of from approximately 3.0 to approximately 6.0.

175. The method of claim 174 wherein the pH of the composition is from approximately 4.0 to approximately 5.0.

176. The method of claim 174 wherein the emulsifier is selected from the group consisting of ammonium lauryl sulfate, sodium laureth sulfate, sodium oleyl succinate, ammonium lauryl sulfosuccinate, sodium dodecylbenzenesulfonate, ammonium laureth sulfate, sodium N-lauryl sarcosinate, and sodium lauryl sulfate.

177. The method of claim 176 wherein the emulsifier is sodium lauryl sulfate.

178. The method of claim 174 wherein the acidic wax is selected from the group consisting of beeswax, carnauba wax, candelilla wax, siliconyl beeswax, siliconyl carnauba wax, and a synthetic acidic wax.

179. The method of claim 178 wherein the acidic wax is beeswax.

180. The method of claim 174 wherein the skin condition or disease is selected from the group consisting of epidermolysis bullosa, decubitus ulcers, pressure ulcers, diabetic ulcers, milia, eczema, urticaria, atopic dermatitis, contact dermatitis, arthritis, gout, lupus erythematosus, acne, alopecia, carcinomas, psoriasis, rosacea, miliaria, skin infections, post-operative care of incisions, post-operative skin care following any variety of plastic surgery procedures, skin care following radiation treatment, care of dry, cracked or aged skin and skin lines.

181. The method of claim 180 where the skin condition or disease is selected from the group consisting of decubitus ulcers, pressure ulcers and diabetic ulcers.

182. The method of claim 174 further comprising administering an additional therapeutic agent in a therapeutically effective quantity.

183. The method of claim 182 wherein the additional therapeutic agent is selected from the group consisting of steroids, nonsteroidal anti-inflammatory agents, leukotriene antagonists, and monoclonal antibodies.

184. The method of claim 174 wherein the composition further comprises at least one of:
(a) an emollient component comprising at least one ingredient selected from the group consisting of lanolin oil, cetyl alcohol, stearyl alcohol, and cod liver oil;
(b) at least one antioxidant selected from the group consisting of butylated hydroxytoluene and butylated hydroxyanisole;
(c) a preservative component comprising at least one preservative selected from the group consisting of methylparaben and propylparaben;
(d) tetrasodium EDTA; and
(e) a solvent component comprising at least one solvent selected from the group consisting of ethylene glycol, propylene glycol, butylene glycol, and glycerin.

185. The method of claim 175 wherein the composition comprises an oil-in-water emulsion comprising:
water;
sodium lauryl sulfate;
propylene glycol;
tetrasodium EDTA;
citric acid;
lanolin oil;
cetyl alcohol;
stearyl alcohol;
an acidic wax selected from the group consisting of beeswax, carnauba wax, candelilla wax, siliconyl beeswax, siliconyl carnauba wax, and a synthetic acidic wax;
cod liver oil;
butylated hydroxytoluene;
methylparaben;
propylparaben;
greater than 2.0% allantoin; and
fragrance.

186. The method of claim 185 wherein the acidic wax is beeswax.

187. The method of claim 185 wherein the composition comprises:
from about 50% to about 90% of water;
from about 0.5% to about 4.0% of 30% sodium lauryl sulfate;
from about 2.0% to about 9.0% of propylene glycol;
from about 0.05% to about 0.5% of tetrasodium EDTA;
from about 0.05% to about 0.5% of citric acid;
from about 5% to about 15% of lanolin oil;
from about 1% to about 10% of cetyl alcohol;
from about 1% to about 5% of stearyl alcohol;
from about 0.5% to about 5% of an acidic wax selected from the group consisting of beeswax, carnauba wax, candelilla wax, siliconyl carnauba wax, and beeswax, siliconyl carnauba wax, and a synthetic acidic wax;
from about 1.0% to about 7.0% of cod liver oil;
from approximately 0.1% to approximately 1.0% of butylated hydroxytoluene;
from approximately 0.1% to approximately 0.5% of methylparaben;
from approximately 0.1% to approximately 0.5% of propylparaben;
from greater than 1.0% to approximately 12.0% of allantoin; and
from approximately 0.05% to approximately 0.5% of fragrance.

188. The method of claim 187 wherein the acidic wax is beeswax.

189. The method of claim 187 wherein the composition comprises:
approximately 67% of water;
approximately 2.5% of sodium lauryl sulfate;
approximately 5.7% of propylene glycol;
approximately 0.15% of tetrasodium EDTA;
approximately 0.09% of citric acid;
approximately 10.6% of lanolin oil;
approximately 3.5% of cetyl alcohol;
approximately 1.7% of stearyl alcohol;
approximately 2.5% of an acidic wax selected from the group consisting of beeswax, carnauba wax, candelilla wax, siliconyl beeswax, siliconyl carnauba wax, and a synthetic acidic wax;
approximately 2.0% of cod liver oil;
approximately 0.5% of butylated hydroxytoluene;
approximately 0.3% of methylparaben;
approximately 0.25% of propylparaben;
approximately 3.00% of allantoin; and
approximately 0.20% of fragrance.

190. The method of claim 189 wherein the acidic wax is beeswax.
